(19) United States
(12) Patent Application Publication
(10) Pub. No.: US 2024/0329243 A1
(43) Pub. Date: Oct. 3, 2024

(54) MIXED ARRAY IMAGING PROBE

(71) Applicant: DeepSight Technology, Inc., Santa Clara, CA (US)

(72) Inventors: Yihang Li, St. Louis, MO (US); Linhua Xu, University City, MO (US); Joshua Arnone, St. Peters, MO (US); Michael Hazarian, San Jose, CA (US); Haochen Kang, Sunnyvale, CA (US); Danhao Ma, San Jose, CA (US); Lan Yang, Clayton, MO (US); Danhua Zhao, San Jose, CA (US); Jiangang Zhu, St. Louis, MO (US)

(21) Appl. No.: 18/597,493

(22) Filed: Mar. 6, 2024

Related U.S. Application Data
(60) Provisional application No. 63/450,554, filed on Mar. 7, 2023.

(51) Int. Cl. G01S 15/89; A61B 5/00; B06B 1/06

(52) U.S. Cl. CPC G01S 15/8965; A61B 5/0097; B06B 1/0607

(57) ABSTRACT

An apparatus for imaging a target and a process of making the apparatus are provided. The apparatus includes a housing and a distal portion. The distal portion includes an acoustic subarray on a first substrate configured to transmit acoustic signals toward the target. The distal portion includes an optical subarray on a second substrate, configured to detect acoustic signals from the target. The distal portion includes an input/output (I/O) region including one or more optical I/O channels. The one or more optical I/O channels is configured to bend optical signals between the optical subarray and the one or more optical I/O channels.

### CROSS-REFERENCE TO RELATED APPLICATIONS

This application claims the benefit of U.S. Provisional Patent Application No. 63/450,554, filed on Mar. 7, 2023, titled “Mixed Array Imaging Probe,” which is hereby incorporated by reference in its entirety.

### FIELD OF THE INVENTION

The present disclosure relates to optical sensing, and without limitation to a design and packaging of an optical-acoustic mixed ultrasound imaging probe.

### DESCRIPTION OF RELATED ART

Acoustic or ultrasound imaging technology is used in various industries, particularly in non-invasive measurements, remote sensing, and medical imaging. Acoustic imaging technology operates by transmitting acoustic signals toward an object and detecting resulting echo signals that reflect or generate from the object in response to the transmitted acoustic signals. Ultrasound, a form of non-ionizing radiation, is an advantageously non-invasive form of imaging. The resolution of ultrasound increases by transmitting higher frequency acoustic waves. However, the depth of penetration decreases due to the increased acoustic attenuation. This tradeoff between resolution and penetration depth poses a challenge.

A conventional ultrasound imaging probe consists of a cable, cable strain relief, a proximal, ergonomic enclosure, and a distal nosepiece, along with the transducer to transmit and receive acoustic signals, with the signals then being processed to produce imaging.

Various known ultrasound transducers used in imaging have numerous drawbacks. For example, some ultrasound transducers are made of piezoelectric material, such as lead zirconate titanate (PZT), polymer thick film (PTF), and polyvinylidene fluoride (PVDF). However, some of the challenges associated with use of piezoelectric properties of these materials include high operation voltage requirements, a high electric field requirement (which may cause breakdown and failure), a non-linear response with high hysteresis, and limited angle of detection. The bandwidth of these materials is also limited. PZT materials with 6 dB bandwidth can generally reach only about a bandwidth of 70%. Certain composite PZT materials have a slightly increased bandwidth, but still only achieve up to about a bandwidth of 80%. As another example, single crystal materials have increasingly been used in an effort to improve performance of ultrasound probes but have lower Curie temperatures and are brittle. Another type of transducer material is silicon, which can be processed to build Capacitive Micromachined Ultrasound Transducer (CMUT) probes that can have increased bandwidth. However, CMUT probes are not very sensitive or reliable. Moreover, CMUT probes have several operational limitations. For example, CMUT probes are nonlinear transducers and, therefore, are not generally suitable for harmonic imaging. In addition, CMUT probes require an additional bias voltage to operate properly. Thus, there is a need for new and improved devices and methods for ultrasound sensing.

### SUMMARY

Various examples and embodiments are described in relation to a mixed array probe. These illustrative examples are mentioned not to limit or define the scope of this disclosure, but rather to provide examples to aid understanding thereof. Illustrative examples are discussed in the Detailed Description. Advantages offered by various examples may be further understood by examining this specification.

### BRIEF DESCRIPTION OF DRAWINGS

The accompanying drawings, which are incorporated into and constitute a part of this specification, illustrate one or more certain examples and, together with the description of the example, serve to explain the principles and implementations of the certain examples.

FIG. 1A shows a perspective drawing of an ultrasound imaging probe, according to this disclosure.

FIG. 1B depicts an example internal mixed array in the example ultrasound imaging probe in FIG. 1A.

FIG. 1C is a block diagram illustrating one example of a mixed array in an ultrasound imaging probe.

FIG. 1D depicts part of the mixed array support structure of the example ultrasound imaging probe in FIG. 1A.

FIG. 1E depicts another view of the mixed array support structure of the example ultrasound imaging probe in FIG. 1A.

FIG. 2A is an example optical I/O design based on a mirror coating with an interposer chip.

FIG. 2B is an example optical I/O design based on a straight fiber array.

FIG. 2C is an example optical I/O design based on a vertical interposer chip with a straight fiber array.

FIG. 2D is an example optical I/O design based on a vertical interposer chip with a mirror structure.

FIG. 2E is an example optical I/O design based on a mirror coating without an interposer chip.

FIG. 2F is an example optical I/O design based on an interposer chip mating with a fiber array.

FIG. 3 shows another view of FIGS. 2A and 2E with additional details, according to one embodiment.

FIG. 4 shows another view of FIG. 2D, illustrating an angle-polished interposer with mirror coating, according to one embodiment.

FIG. 5 is another view of FIG. 2B with a 2D surface coupler array and multi-core fibers in a fiber array, according to one embodiment.

FIG. 6A is an illustration of a reflective focusing right-angle optical fixture, according to one embodiment.

FIG. 6B is an example fabrication process of the reflective focusing right-angle optical fixture in FIG. 6A, according to one embodiment.

FIG. 7 is an illustration of a refractive focusing right-angle optical fixture with a lens plate, according to one embodiment.

FIG. 8 is an illustration of a design based on photonics wire-bonding techniques in a probe, according to another embodiment.

FIG. 9A is an example electrical I/O design including 1D pad arrays for wire-bonding a PIC chip and an FPC.

FIG. 9B is another example electrical I/O design including 1D pad arrays for wire-bonding the PIC chip and the FPC.

FIG. 9C is yet another example electrical I/O design including 1D pad arrays for wire-bonding the PIC chip and the FPC.

FIG. 9D is an example electrical I/O design including 2D pad arrays for bonding the PIC chip and the FPC via flip chip bonding.

FIG. 9E is another example electrical I/O design including 2D pad arrays for bonding the PIC chip and the FPC via flip chip bonding.

FIG. 9F is another example electrical I/O design including 2D pad arrays for bonding the PIC chip and the FPC via flip chip bonding.

FIG. 10A is an illustration of the design of an AEG module, according to one embodiment.

FIG. 10B is an illustration of a conventional design of an AEG module, according to one embodiment.

FIG. 10C is an illustration of the fabrication of an AEG module, according to one embodiment.

FIG. 10D is an illustration of a conventional fabrication of an AEG module, according to one embodiment.

FIG. 11 illustrates a double dicing process, according to one embodiment.

FIG. 12 illustrates an alternative design of the PIC module package, according to one embodiment.

FIG. 13 shows a cone-shaped nosepiece design, according to one embodiment.

FIG. 14 shows an alternative design of a mixed ultrasound imaging probe, according to one embodiment.

FIG. 15A is a schematic illustration of modularized optical subarrays arranged in a stair pattern with individual backing blocks, according to one embodiment.

FIG. 15B is a schematic illustration of modularized optical subarrays arranged in a stair pattern sharing a unified backing block embedded in a thermomechanical substrate, according to one embodiment.

FIG. 15C is a schematic illustration of modularized optical subarrays arranged in a polygonal pattern with individual backing blocks, according to one embodiment.

Similar reference characters denote corresponding features consistently throughout the attached drawings.

### DETAILED DESCRIPTION

The following various structures are described herein according to their geometric properties. As discussed herein, structures so described may vary from the described shape according to the tolerances of known manufacturing techniques. Unless otherwise specified, features described with the term “substantially” are understood to be within 5% of exactness. For example, features described as “substantially parallel” may deviate from true parallel by 5%.

Ultrasound or acoustic imaging technology, a form of non-ionizing radiation, is an advantageously non-invasive form of imaging used in various industries, particularly in non-invasive measurements, remote sensing, medical imaging, diagnostic procedures, surgical procedures, and therapeutic procedures. In medical imaging, diagnostic, surgical and therapeutic applications, the clinician uses ultrasound to image internal structures of patients, including tissue, organ, bone, and other anatomical structures, implants, medical tools, or other objects within the insonified region.

Some existing imaging technologies use acoustic energy generating (AEG) materials for transducers to generate and receive acoustic signals. Commonly used AEG transducers include piezoelectric materials, such as lead-zirconate-titanate (PZT), ceramic, piezoelectric single crystal (e.g. PIN-PT, PIN-PMN-PT), polymer thick film (PTF), polyvinylidene fluoride (PVDF), capacitive micromachined ultrasonic transducers (CMUT), photoacoustic transducers, and piezoelectric micromachined ultrasound transducers (PMUT), among many other materials known to those of skill in the art. However, some of the challenges associated with use of these materials, aside from the trade-offs between resolution and penetration depth, include high operation voltage requirements, a high electric field requirement (which may cause breakdown and failure), a non-linear response with high hysteresis, and limited angle of detection. Furthermore, the detection sensitivity of AEG transducers is a function of size, thereby limiting the suitability of size-constrained applications, for example intravascular ultrasound (IVUS) devices.

Another challenge is the narrow bandwidth of an AEG transducer. For example, for ultrasound transducers made of piezoelectric material, such as lead zirconate titanate (PZT), the 6 dB bandwidth of PZT is generally limited to only about 70%. Certain composite PZT materials have a slightly increased bandwidth, but still only achieve a bandwidth of up to about 80%. As another example, single crystal materials have increasingly been used to improve performance of ultrasound probes but have lower Curie temperatures and are brittle. Another type of transducer material is silicon, which can be processed to build Capacitive Micromachined Ultrasound Transducer (CMUT) probes that can have increased bandwidth. However, CMUT probes are not very sensitive or reliable. Moreover, CMUT probes have several operational limitations. For example, CMUT probes are nonlinear transducers and, therefore, are not generally suitable for harmonic imaging. In addition, CMUT probes require an additional bias voltage to operate properly. Additionally, some AEG transducers and systems may be affected by electromagnetic interference, such as that caused by ablation tools, cauterization tools, or any other procedure or technique that applies electrical energy to tissue. Furthermore, use of an electro-mechanical transducer at the distal end can include an electrically conductive line and associated components requiring additional design and safety requirements and challenges. Thus, there is a need for new and improved devices and methods for ultrasound imaging modes with various frequency harmonics to obtain higher resolution, better penetration, and fewer artifacts than fundamental imaging of conventional ultrasound sensing.

Photonic devices and optical pressure detection techniques have shown great promise for ultrasound detection. In photonic devices, refractive index modulation and/or shape deformations due to strain induced by an acoustic wave are translated into changes in the intensity of the detected light or the spectral properties of the device. In some existing devices, optical resonators have been used as highly sensitive ultrasound detectors. In general, the performance of an optical resonator is limited by its quality factor Q (i.e., the higher the Q, the lower the optical loss and the smaller the detectable resonance shift), the optical insertion loss of the whole optical path as well as by the acousto-optical and mechanical properties of the material from which the resonator is made. Optical sensors, for example interference based optical sensors, optical resonators, and interferometers, may have high sensitivity, broad bandwidth and wide acceptance angle in reception of ultrasound signals, compared to other types of ultrasound sensors. Because of the high sensitivity, broad bandwidth and wide acceptance angle of optical sensors, the image produced by the optical sensors may have improved spatial resolution, improved penetration depth, improved signal-to-noise ratio (SNR), improved tissue harmonic imaging, and/or improved Doppler sensitivity.

The optical sensors may be coupled to a light source, transmit light, and be useful in practice (e.g., for an ultrasound imaging or other transducing application in an acousto-optic system). Acousto-optic systems based on optical sensors may directly measure ultrasonic waves through the photo-elastic effect and/or physical deformation of the resonator(s) in response to the ultrasonic waves (e.g., ultrasonic echoes). For example, in the presence of ultrasonic (or other pressure) waves, the transmission or reflective spectrum of optical resonator may undergo a spectral shift caused by changes in the refractive index and shape of the optical resonator. The spectral change can be monitored and analyzed in spectral domain and light transmission intensity to and from the optical resonator. Additional spatial and other information can furthermore be derived by monitoring and analyzing transmitted or reflected light among multiple optical resonators. Furthermore, other physical parameters, for example temperature and pressure, can affect the transmitted light providing additional information to support multi-dimensional sensing. However, challenges exist in designing a robust sensor with acceptable Q value with minimum optical insertion loss in the whole optical path in a form factor used in ultrasound imaging.

In some configurations, a plurality of transducer types is used. In some examples, the ultrasound array may include the same type of elements. Alternatively, the ultrasound array may include different types of elements. For example, a probe may include one or more AEG transducers, such as one or more of a piezoelectric transducer, a PZT transducer, a PTF transducer, a PVDF transducer, a CMUT, a PMUT, a photoacoustic transducer, a transducer based on single crystal materials (e.g., LiNb03(LN), Pb(Mg113Nb213)-PbTiQ3 (PMN-PT), and Pb(In112Nb112)-Pb(Mg113Nb213)PbTiQ3 (PIN-PMN-PT)), combinations thereof, and the like.

Additionally, in some examples, the ultrasound array may include one or more optical sensors, such as an interference-based optical sensor, which may be one or more optical interferometers and/or optical resonators, or a sensor array for beamforming to construct high quality ultrasound images of targets or areas of interest as noted. Optical resonators may have high sensitivity, broad bandwidth, and wide acceptance angle in reception of ultrasound signals, compared to other types of ultrasound sensors. The one or more array elements of a first type (e.g., AEG transducers) may be used to form a first image. In parallel, the one or more array elements of a second type (e.g., the optical sensors) are used to detect acoustic signals that can be used to form a second image. The second image generated by these highly sensitive and broadband optical sensors may be used independently or can be combined with the first image to form an even further improved image. In some configurations, the optical sensors can be used independently of a transmit element or transmit array. Diagnostic and therapeutic procedures may use additional information from the sensed signals beyond the image, for example when used for multi-dimensional sensing.

An optical sensor may perform multi-dimensional sensing (e.g., to measure a plurality of different physical signals substantially simultaneously in real-time or near real-time). An optical sensor system may generally include one or more optical sensors where an optical sensor (e.g., single sensor) may be used to detect multiple physical signals, such as temperature, pressure, acoustic waves, and the like, by analyzing sensor responses, such as a mode shift (e.g., change in frequency, depth, shape of spectral response), a baseline drift, a mode split, and a mode broadening.

A sensor signal may be used to generate a plurality of measurement signals corresponding to a plurality of physical signals. A multi-dimensional sensor capable of measuring a plurality of physical signals. Accordingly, while an array may include multiple optical sensors, one or more optical sensors in the array may function independently and singularly from the other optical sensors in the array.

The present disclosure generally relates to the field of ultrasound, and particularly to methods and devices that enable ultrasound transducing using a mixed array including, for example integrating an array of optical sensors and other transducers.

Generally, in some embodiments, an apparatus for imaging a target may include an ultrasound transducer array that includes one or more array elements of a first type and one or more array elements of a second type different from the first type. The first type may be a transducer (e.g., AEG materials including, for example, piezoelectric transducers or CMUTs) configured to transmit acoustic waves, and the second type may be an optical sensor (e.g., an interference-based optical sensor such as an optical resonator, an optical interferometer, etc.). The array elements of the first and second types are configured to detect acoustic echoes corresponding to the transmitted acoustic waves. Alternatively, the array elements of the first type may only be configured to transmit acoustic waves.

The following commonly owned patent applications disclose various methods and systems for optical sensors, mixed array transducers, ultrasound beamforming and image processing, the disclosures of which are incorporated by reference for all purposes: U.S. application Ser. No. 17/832,507, filed Jun. 3 2022, titled Whispering Gallery Mode Resonators for Sensing Applications; U.S. application Ser. No. 17/956,640, filed Sep. 29, 2022, titled Optical Microresonator Array Device for Ultrasound Sensing; U.S. application Ser. No. 18/091,073, filed Dec. 29, 2022, titled Acousto-Optic Harmonic Imaging With Optical Sensors; U.S. application Ser. No. 17/990,596, filed Nov. 18, 2022 and titled Mixed Ultrasound Transducer Arrays; U.S. application Ser. No. 17/244,605, filed Apr. 29, 2021, titled Modularized Acoustic Probe; U.S. application Ser. No. 18/032,953, filed Apr. 20, 2023, titled Image Compounding for Mixed Ultrasound Sensor Array; U.S. application Ser. No. 18/025,081, filed Mar. 7, 2023, titled Synthetic Aperture Imaging Systems and Methods Using Mixed Arrays; U.S. application Ser. No. 18/091,073, filed Dec. 29, 2022, titled Acousto-Optic Harmonic Imaging with Optical Sensors; PCT Application c, filed Oct. 7, 2022, titled Ultrasound Beacon Visualization with Optical Sensors; PCT Application PCT/US2022/041252, filed Aug. 23, 2022, titled Multi-Dimensional Signal Detection with Optical Sensor; and U.S. Provisional Application 63/550,515, filed Feb. 6, 2024, titled Photonic Integrated Acoustic Sensor.

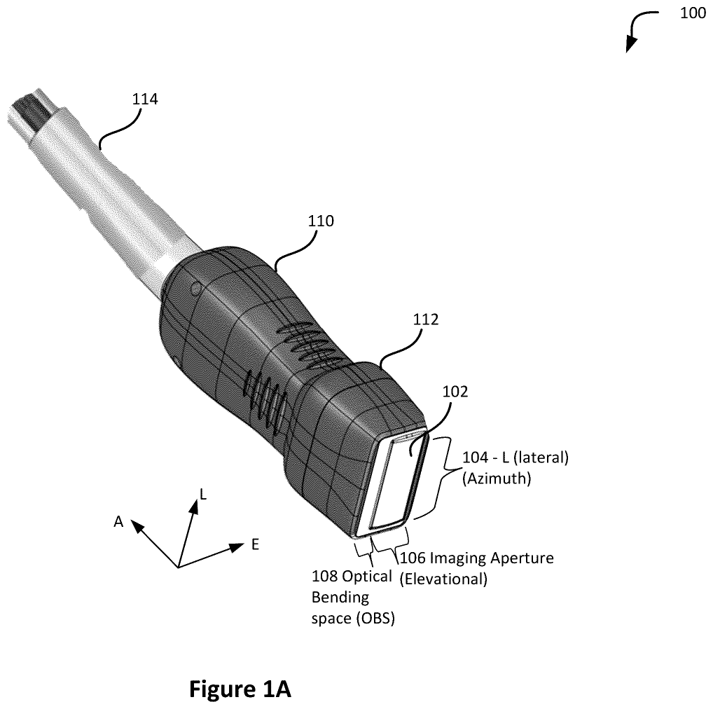

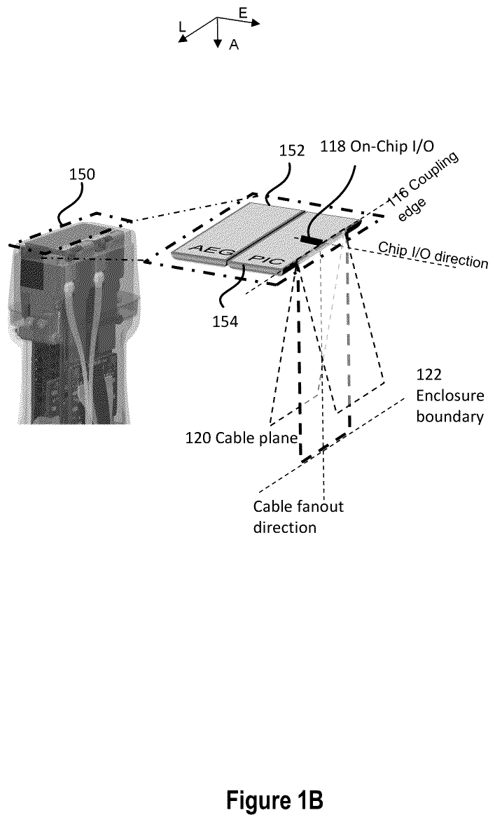

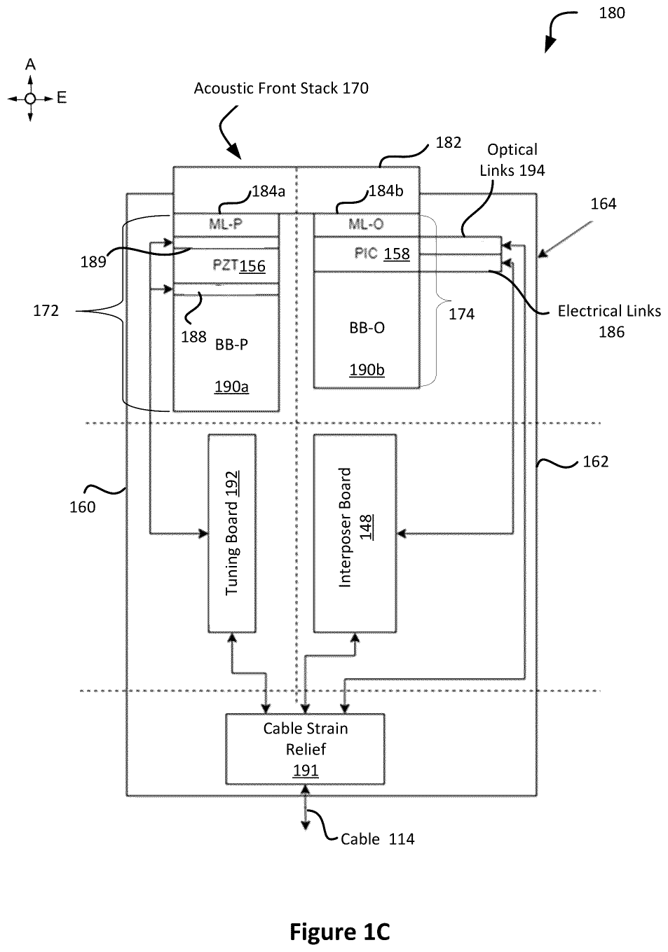

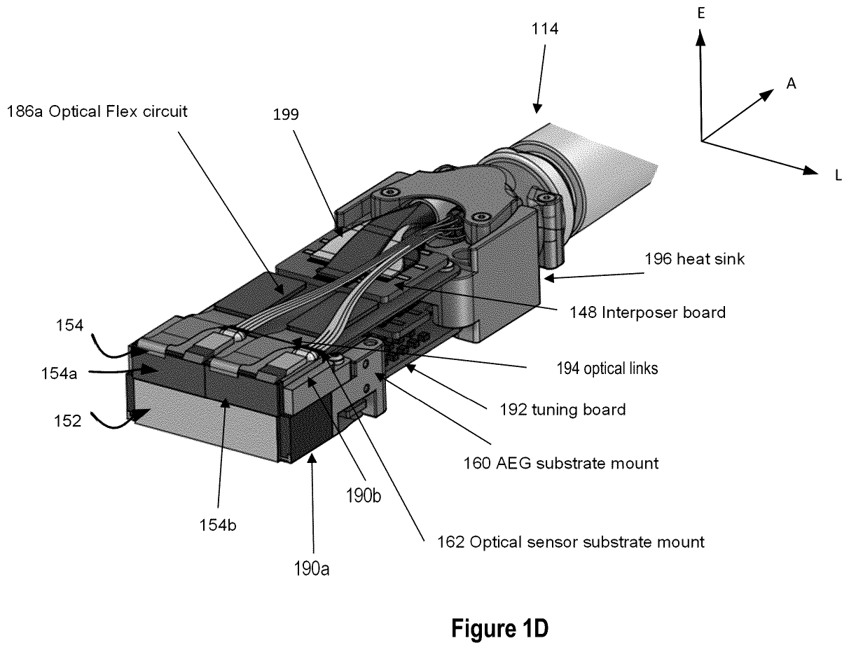

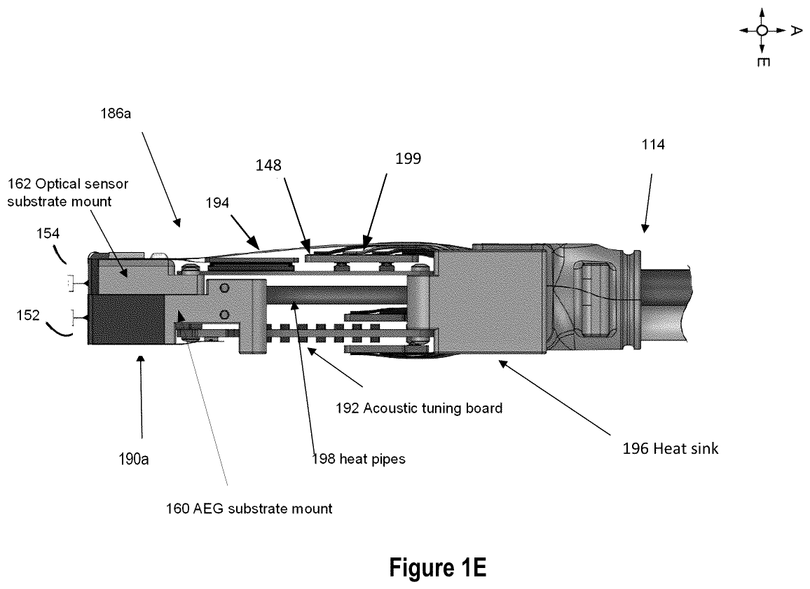

FIG. 1A shows a perspective drawing of an ultrasound imaging probe, according to this disclosure. FIG. 1B depicts internal mixed array 150 in the example probe in FIG. 1A. FIG. 1C is a block diagram 180 illustrating one example of a mixed array in an ultrasound imaging probe. FIG. 1D depicts part of the mixed array support structure of the example ultrasound imaging probe in FIG. 1A. FIG. 1E depicts another view of the mixed array support structure of the example ultrasound imaging probe in FIG. 1A.

As shown in FIG. 1A, the geometry of an ultrasound imaging probe could be described in the following three dimensions: lateral (L), elevational (E) and axial (A). In some embodiments, the ultrasound imaging probe includes a mixed array of an AEG material subarray and a photonic integrated circuit (PIC) receiver subarray (optical subarray for short), which will be described in detail in FIGS. 1B and 1C. The front end of the probe, which contacts the surface of the imaging target, can be mostly occupied by the acoustic or imaging aperture 102 (e.g., acoustic front stack in FIG. 1C). The lateral length 104 of the acoustic aperture is generally determined by AEG array parameters (e.g., element count, element width, element pitch, gap between AEG elements (e.g., Kerf)). The probe elevation dimension consists of the elevation width 106 of the acoustic aperture, determined by the geometry of the single-element transmitters and in some embodiments, an optical bending space 108 needed to accommodate components of the optical sensor subarray as will be explained in further detail. The outer portion of the probe consists of a housing 110, nosepiece 112 and cable 114.

FIG. 1B illustrates an enlarged section of the face of a mixed array 150 with the orientation of certain optical array components. Mixed array 150 is shown as a linear array. The elements of the AEG subarray 152 and optical subarray 154 form the basic building block of an ultrasound probe transducer and sensor. Mixed array 150 may be characterized by a lateral pitch, determined by the imaging application constraints (for example, a lateral pitch that is equal to or less than an acoustic wavelength in tissue); lateral width (L_{w}); and elevation width (Wₑₗₑ) determined by a desired acoustic focus of the ultrasound probe. Acoustic focus (e.g., focus in the elevation dimension) can apply to both transmitting and receiving process. The focal depth is determined by the elevation size (Wₑₗₑ) and/or an acoustic lens. The lateral pitch of single elements adds up across the array and forms a lateral imaging aperture with a length Lₗₐₜ, which can at least partially determine an imaging performance of the array.

A higher-dimensional array can be formed by combining multiple 1-dimensional (1D) arrays. For example, the array can be configured for operation in a 1.25-dimensional (1.25D) array configuration, a 1.5-dimensional (1.5D) array configuration, a 1.75-dimensional (1.75D) array configuration, a 2-dimensional (2D) array configuration, or another array configuration. Generally, dimensionality of the ultrasound transducer array relates to the range of elevation beam width (or elevation beam slice thickness) that is achievable when imaging with the ultrasound transducer array, and how much control the system has over the transducer array's elevation beam aperture size, foci, and/or steering throughout an imaging field (e.g., throughout imaging depth). A 1D array has one row of elements in elevation dimension and a fixed elevation aperture size. A 1.25D array has multiple rows of elements in elevation dimension and a variable elevation aperture size, but a fixed elevation focal point via an acoustic lens. A 1.5D array has multiple rows of elements in elevation dimension, a variable elevation aperture size, and a variable elevation focus via electronical delay control. A 1.75D array is a 1.5D array with additional elevation beam steering capability. A 2D array has a number of elements in both lateral and elevation dimensions to satisfy a minimum pitch design constraint for large beam steering angles. The array may have a geometric shape or other shape to accommodate various types of non-linear probes, such as, but not limited to curvilinear, convex, and/or phased array.

As shown in FIG. 1C, an AEG subarray 152 includes PZT modules 156 and an optical subarray 154 includes PIC modules 158. The AEG and optical subarrays 152 and 154 are mechanically supported and protected by a thermomechanical substrate and enclosure. AEG subarray 152 can be mounted to a suitable substrate separate from the optical subarray substrate. Alternatively, the AEG subarray and the optical PIC subarray may be mounted to a single substrate. The PIC and AEG array modules may be formed as a single unit or may consist of several modules.

An acoustic front stack 170 is on the distal end of the probe and includes acoustic stack 172 and optical stack 174. The acoustic front stack 170 also includes an interface layer 182 contacting the surface of the area to be imaged and transmitting acoustic signals between the probe and the imaging target. Interface layer may be formed separate or as a component of the acoustic stack 172 and/or optical stack 174. The optical stack 174 may be disposed behind or within interface layer 182. The interface layer 182 can be made from a biocompatible material with minimal acoustic impedance that can also serve as a moisture barrier and electrical insulator. In some embodiments, an electrical insulating and chemical resistance layer (e.g., parylene) may be placed between the outer surface of the interface layer 182 and the AEG subarray 152 to ensure electrical safety. In some embodiments, the interface layer 182 may be a single integrated component of multiple different materials or may be a single integrated component of a single material. The interface layer 182 can include acoustic lens, acoustic windows, sealing and bonding layers, etc.

The interface layer 182 may further attached to an acoustic matching layer selected for acoustic impedance matching with a target environment, to reduce acoustic reflections at the interface between the mixed array and the target environment. As shown in FIG. 1C, in an embodiment each subarray has its own matching layer with matching layer ML-P 184a on the AEG subarray 152 and matching layer ML-O 184b on the optical subarray 154. In some embodiments, the optical subarray 154 may not require a matching layer due to the acoustic impedance of the materials comprising the optical sensors.

In embodiments, the interface layer 182 may further be configured to include one or more acoustic lenses to assist in focusing/steering transmitted acoustic signals and collimating the wavefront of received acoustic signals. An acoustic lens is used to focus the ultrasound beam on a plane perpendicular to the imaging plane or axial-elevation plane. The acoustic lens typically consists of materials with acoustic impedance close to human tissue. With a specific geometry, the lens provides an appropriate slice thickness to enable uniform sensitivity and improved SNR across field of view. Room-Temperature-Vulcanizing (RTV) silicone is a typical acoustic lens material, because a sub-mm RTV layer may provide good electrical and moisture isolation as well as enough durability as the transducer-patient contact surface. In some embodiments, parylene, an electrical insulating and chemical resistance layer may be placed between the lens and the AEG elements to ensure electrical safety. The acoustic lens can be over-molded directly onto the acoustic stack 172 and/or the optical stack 174 with nosepiece 112 in place. The thickness of the lens can be controlled using precise fixturing, where potential tolerance stack-ups of individual components can be designed before the precise fixturing process. The acoustic lens, in addition to the interface layer and/or parylene layer, can hermetically seal the acoustic window or aperture 102 in the nosecone of nose piece 112.

It should be noted that the acoustic front stack in front of the AEG subarray 152 and the optical subarray 154 can include different stacks, known as mixed acoustic front stack. In some embodiments, there is an axial offset between the optical receiving plane (e.g., top of the ML-O 184b) and the AEG plane (e.g., top of ML-P 184a) due to transducer designs or mechanical interface with other building blocks. An acoustic window made with materials with low acoustic attenuation and good acoustic match with top and bottom layers (e.g., flexible or rigid elastomers, with or without matching layers on top and bottom sides) could be introduced to bridge the offset. In some embodiments, optical receivers with small elevation size (<1 mm) do not necessarily need acoustic lens. In both cases, a casted RTV layer could be introduced for top finish, whose shape could be designed to serve as the acoustic lens if needed. The design of a mixed acoustic front stack can follow one or more of the following: (1) The top finish of the acoustic stack can have a non-concave and smooth shape with sufficient electrical and moisture isolation and durability; (2) The acoustic front stack may not introduce significant acoustic attenuation among all acoustic frequency band of interest.

The interface layer 182 may also include a couplant made of a material with low attenuation and impedance matching such as a flexible or rigid elastomer. The interface layer 182 may be a single or multiple piece component attached via adhesive and/or may be molded in place to the AEG and optical sensor arrays. In some embodiments, the interface layer 182 may be disposed within or as part of a transducer housing as an exterior layer of the transducer device between the AEG and optical sensor arrays and a surrounding environment. Further, the interface layer 182 provides protection for the optical sensors.

Matching Layers (MLs) (e.g., ML-P 184a and ML-O 184b) connect the interface layer 182 with PZT array modules forming acoustic stack 172 and PIC array modules forming optical stack 174, which are electrically and/or optically connected to a cable 114 via electrical boards, optical links, and a cable strain relief in the rear part of the probe. In some embodiments, the MLs may be optional if a loss of acoustic power reaching the AEG receiver and/or optical sensors is tolerable. In this way, the potential bandwidth limits brought by MLs can be removed. Inside the probe, electrical links 186, which can also be called electrical I/O channels, can be realized by flexible printed circuits (FPC), and optical links, which can also be called optical I/O channels, can be realized by optical fibers, interposer chips, printed optical waveguides, etc. Both the PZT and PIC modules 156 and 158 are directly mounted on backing blocks (e.g., BB-P 190a and BB-O 190b), which can provide mechanical support to the modules and serve as an acoustic absorber that (1) may or may not match the acoustic impedance, depending on the level of damping desired, of the PIC modules 158 and/or PZT modules 156, respectively, and (2) can significantly attenuate the energy of the acoustic wave.

The practice of ultrasound imaging can introduce a number of design considerations to the probe design and packaging process. For example, innovative solutions include techniques to accommodate both acoustic and optical subarrays within a handheld probe having a size and shape of a conventional ultrasound probe, along with ergonomic requirements, to reinforce the probe and the subarrays to withstand forces exerted on the device during use, and to ensure that any thermal energy generated by the PIC and PZT modules are efficiently dissipated into the ambient air without discomfort to the patient or operator.

Operating such a mixed array ultrasound probe uses a compact nosepiece 112, characterized by a small total elevation length of the front end and a total input/output (I/O) region width comparable to the azimuthal width of the sensor array region. The packaging design of the PIC modules 158 follows one or more of the following considerations: (1) minimize the I/O channel counts and the I/O pitch; (2) minimize the physical size of the assembly by routing fibers and electrical wires such that they bend backwards sharply outside the chip. The embodiments described herein address these design considerations.

The nature of the mixed array introduces a geometric gap between the two subarrays, and minimizing this gap could benefit both the imaging quality and the compact elevation footprint of the probe. In addition, for practical applications, it may be desirable that the nosepiece in the E-direction is minimized, so that the overall probe elevation at the patient interface approaches that of the acoustic aperture. However, it should be noted that opto-acoustic sensors (e.g., chip-based sensors) usually function with in-plane (E-L plane in probe coordinate) optical and electrical signals. Thus, a sharp substantially right-angle bending of both optical and electrical signal can prevent introducing extra structures in the nosepiece that may otherwise greatly increase the elevation size of the nosepiece and the patient interface of the probe. As shown in FIGS. 1A and 1B, an optical bending space 108 space is needed for accommodating the electrical and optical signals for the optical subarray 154.

In the optical subarray 154, the optical and electrical signals travel within the plane of the chip, or E-L plane as shown in FIG. 1B. In some embodiments (e.g. typical hand-held linear and curvilinear mixed array probes), on-chip optical and electrical I/Os are on one coupling edge 116 of the chip that is substantially in a lateral direction. The on-chip I/O structure 118, especially optical couplers, should be substantially perpendicular to the coupling edge to minimize insertion loss. However, the electrical and optical cables inside the transducer bodies are usually arranged within a cable plane 120 that may form a substantially right-angle to E-L plane, shown in FIG. 1B. The optical and electrical cables can be enclosed with this space indicated as enclosure boundary 122 which can be defined by ergonomic requirements of nosepiece 112 and housing 110 and acoustic aperture 102. Within the probe 100, the cables are bundled and arranged with multiple sections of routing including reflection, bended cable and on-chip waveguide routing in a direction as shown in FIGS. 1D and 1E. Cable fanouts may be introduced to bridge the on-chip I/O and the cable bundles along cable fanout direction without occupying too much elevation space (<50% of elevation size of imaging aperture), known as the “sharp bending”. In some embodiments (e.g. typical hand-hold linear and curvilinear mixed array transducers), where the cable plane is substantially perpendicular to the on-chip I/O structure 118 substantially along the elevation direction and the enclosure boundary defined by the ergonomic design, the sharp bending is substantially a right-angle bending with a deviation caused either by design or fabrication error.

The axial size of the probe and the design of the housing 110 (i.e., the rear case part or the proximal enclosure of the probe 100) are usually defined by ergonomic considerations. Significant pressure may be applied to the patient's skin with such probes while maintaining agility and acute control for the imaging technician. Thus, the shape, weight, center of gravity, and material selection are each important consideration.

Embodiments of the housing of the probe may incorporate a variety of features. For example, such embodiments may include: a type III anodized aluminum housing with a combination of organic contours, dimples, textures, and over-molded silicone regions. The housing can be high strength and light weight, including clamshells and nosecone for impact resistance. Aluminum may be preferable thermally, mechanically, and/or chemically to other materials. Thinner walls can be used than traditional plastic components to reduce size, especially given larger internal electronics in the current design. Tactile ridges can be added to enhance grip. Type III anodization can insulate the probe electrically and provides a very tough, scratch-resistant surface. The type III anodized aluminum provides for another layer for electrical insulation.

Embodiments may include some or all of the following components and structures. First, an embodiment may include a mounting block 160 for an AEG subarray 152. The AEG subarray 152 could be but is not limited to a PZT (the example shown in FIGS. 1C, 1D and 1E), single crystal transducer (SCT), CMUT, PMUT or other known AEG transducers. The mounting block is a light weight, relatively high strength thermal conductor (e.g., aluminum). The mounting block 160 acts as a thermomechanical substrate for the AEG subarray 152 and provides mechanical stability. The mounting block may include integrated thermoelectric coolers and thermistors (or thermocouples) for closed loop control. And an embodiment may utilize a thermally conductive epoxy for attaching the AEG module. The PZT modules 156 shown in FIGS. 1C, 1D and 1E is electrically connected to a tuning board 192 via a flexible printed circuit 188 or a conductor layer 189, which conditions and transmits the signals from the PZT modules 156 through a cable.

An embodiment also includes a heat sink 196, as shown in FIG. 1D. The heat sink 196 is preferably located distally from sensors and patients. The heat sink is preferably a light weight, relatively high strength thermal conductor (e.g., aluminum). The heat sink presents a relatively large surface area with minimal thermal resistance to the housing (so that energy can be dissipated into ambient air). The heat sink may also include a very thin layer of thermal compound at the housing/heat sink interfaces.

Embodiments may also include a mounting block 162 for optical subarray 154. The mounting block acts as a thermomechanical substrate for the optical subarray 154 and provides mechanical stability where optical links 194 (e.g., fiber array unit (FAU)) and electrical links 186 (e.g., flex circuit) attach to the PIC modules 158. The PIC modules 158 may be formed as a unit on a single substrate or being formed on several substrates to form separate modules, such as modules 154a and 154b shown in FIG. 1D. AEG and optical subarrays 152 and 154 could either share the same mounting block or be mounted on two blocks, as shown in FIG. 1D with firm mechanical fixture and good thermal conductivity. The mounting block 162 for the optical subarray 154 is also preferably a light weight, relatively high strength thermal conductor (e.g., aluminum). The mounting block may include integrated thermoelectric coolers and thermistors (or thermocouples) for closed-loop temperature control. The mounting block contains the BB-O 190b for the PIC, which may be cast in place within a milled pocket. And an interposer board 148 may be fixated directly to the block, to which the PIC flex circuit can connect. A significant difficulty in incorporating both an AEG module and a PIC module in the same ultrasound device is routing the optical signals from the PIC to the interposer board 148 and then through the cable. As can be seen in FIG. 1C, optical and electrical I/O is employed to do so, and example techniques for routing the PIC signals are discussed in more detail below.

An embodiment may also include heat pipes 198, as shown in FIG. 1E. The heat pipes 198 connect the mounting block 162 for the optical subarray 154 to the heat sink 196. Such heat pipes provide for efficient transfer of thermal energy over relatively long distances within tight spaces where forced air convection is not an option. Some embodiments include at least two heat pipes, which improves mechanical stability. Other embodiments may include additional heat pipes to increase thermal efficiency in probes that are not spatially constrained. The heat pipes may be secured within precision-reamed holes in aluminum components using thermally conductive epoxy.

The mounting block 162 for the optical subarray 154 can integrate with the AEG subarray 152 described above by securing it to the mounting block 160, and finally attaching the interposer board 148 to the heat sink 196 at the opposite end. Any mechanical and/or thermal connection can be accomplished using screws and thermal compounds at the interface. Finally, assembly jigs and fixtures are used to ensure proper alignment of the two systems before semi-permanent assembly is complete.

An additional consideration is to minimize any acoustic impedance mismatch between the materials needed in both stacks in order to receive effective signals. Interfaces with large acoustic impedance mismatch can cause waves to be reflected in other directions, scattered, or attenuated. The larger the impedance difference at the junction of two substances is, the greater the energy disruption can become. In view of materials needed for the PIC, such as silicon, the typical matching layer material for AEG may not be suitable. A matching layer, typically a single or multi-layer structure made from loaded epoxy, plastic or other material with acoustic impedance between those of the two layers it bridges, bonded on the front face of the AEG elements can improve the acoustic mismatch between the stiff AEG elements and the soft human tissues. Aluminum, in addition to its thermal and mechanical benefits, has a better impedance to match from the optical standpoint.

In practice, the probe is generally pressed against a surface of the imaging target. The force exerted may be up to 30 pounds or in some cases greater than 30 pounds. Thus, the probe needs to be constructed to sustain such forces in some embodiments.

Further, the PIC sensors may be sensitive to local ambient temperature. Embodiments of a probe may include individual controllers (e.g., thermo-optical phase shifters) to stabilize the array operation. Controlling overall temperature can help (1) increase the robustness of the array operation; (2) save total controlling power; and (3) maintain the surface temperature of the acoustic aperture within an optimal and regulated range for medical usage. Thus, thermal management components 199 can also be internal components associated with the optical subarray 154.

Various methods for producing such probes may be utilized. For example, in one embodiment, during assembly, the AEG subarray 152 and the optical subarray 154 in the nosepiece 112 are fabricated, packaged, and assembled separately. Then, both modules are mounted to the thermomechanical substrate, where both the tuning board 192 and the interposer board 148 are mounted too. The FPC from the modules in the nosepiece 112 are connected to on-board connectors, and the optical cables are fixed with an in-probe strain relief structure. The electrical coaxial cables connecting the two boards and the back-end system could be (1) soldered to the tuning board 192 and the interposer board 148 prior to the probe building, or (2) soldered to a small termination board with a board-to-board connector, which is mated to the connector on the tuning board and the interposer board. Finally, cables are bundled together or separately and over-molded in the cable strain relief structure (e.g., a tube).

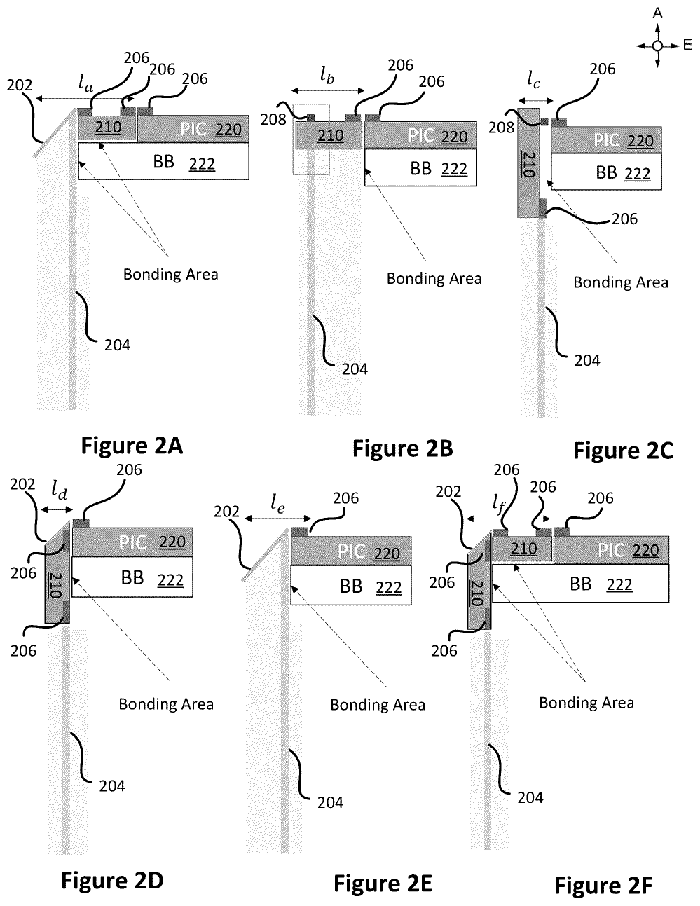

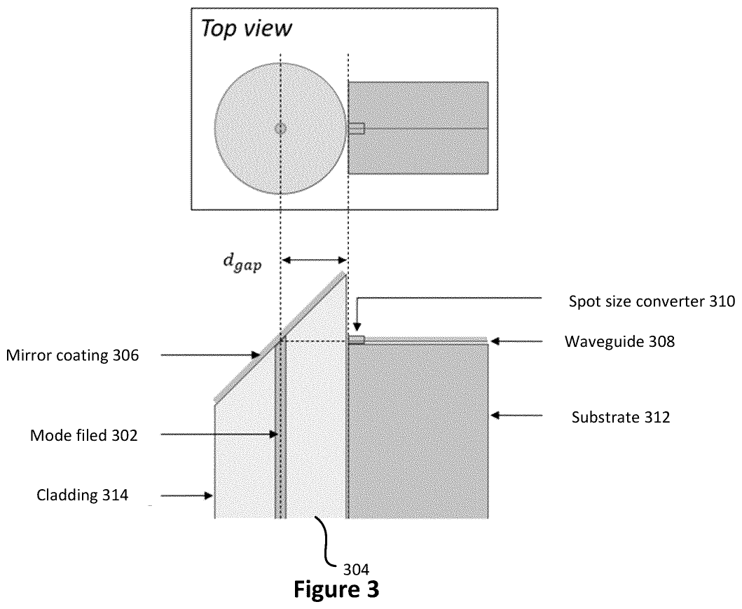

FIGS. 2A-2F show examples of compact optical I/O designs and packaging for example probes. As discussed above with respect to FIGS. 1B and 1C, the sizing considerations of handheld ultrasound mean that the optical I/O, which usually extends the in-plane (E-L plane) optical signal to the side and then bends backward to axial cables, is compact in the elevation direction. The lossless bending radius for normal optical fibers (especially for polarization-maintaining fibers) is limited above ˜5 mm. Thus, a compact optical I/O design based on a mirror layer or surface couplers is proposed, as shown in FIGS. 2A-2F. The bonding area can stabilize the optical coupling against the pressing force during the practice, which is a unique design consideration of the optical packaging in a mixed ultrasound imaging probe. As shown in FIGS. 2A-2F, the optical fiber array is in an axial direction substantially along a cable fanout direction, and is coupled to one or more interposer structures for bending optical signals to/from the optical subarray. The optical fiber array can be mated with an interposer chip in the axial direction including a mirror structure with about 45 degrees in reference to an elevational-lateral plane of the optical subarray via directly edge coupling or another one or more in-plane interposer chips, or one or more surface coupler coupled to the optical subarray. It should be noted that in FIGS. 2A-F, a right-angle bending is assumed. If the design value of the bending angle (angle between chip I/O direction and cable fanout direction in FIG. 1B) deviates from 90 degrees or multi-stage bending is needed, the bending and reflected mirror angle could be adapted to realize the designed bending.

FIG. 2A is an example optical I/O design based on a mirror coating with an interposer chip. As shown in FIG. 2A, the design is based on a mirror coating 202 on a 45-degree polished fiber array 204 that implements a right-angle (e.g., approximately 90 degree) bending. An interposer chip 210 containing spot-size converters is introduced to optimize for the coupling between on-chip optical modes and the in-fiber optical modes after the mirror reflection. The spot-size converter can be optional when the fabrication process of the coupler is compatible with that of PIC chip. Edge couplers 206 are used to couple the interposer chip 210 with the fiber array 204 and the PIC module 220 and BB 222. The distance between the coupling edge of the PIC module and the enclosure of the mixed ultrasound imaging probe, which can be denoted as l, can reflect how compact the mixed ultrasound imaging probe can be with different optical I/O designs. The distance can also present the size of the optical I/O in the elevational direction. With the design in FIG. 2A, lₐ<3 mm could be realized. FIG. 2E shows another example optical I/O design based on a mirror coating without the interposer chip.

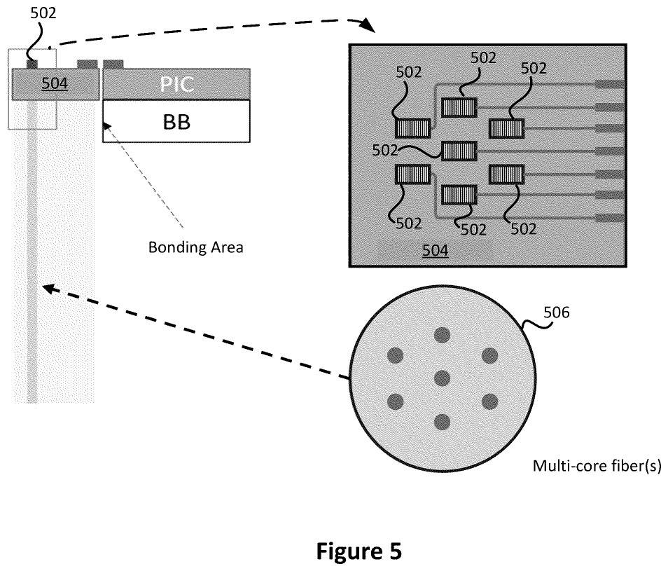

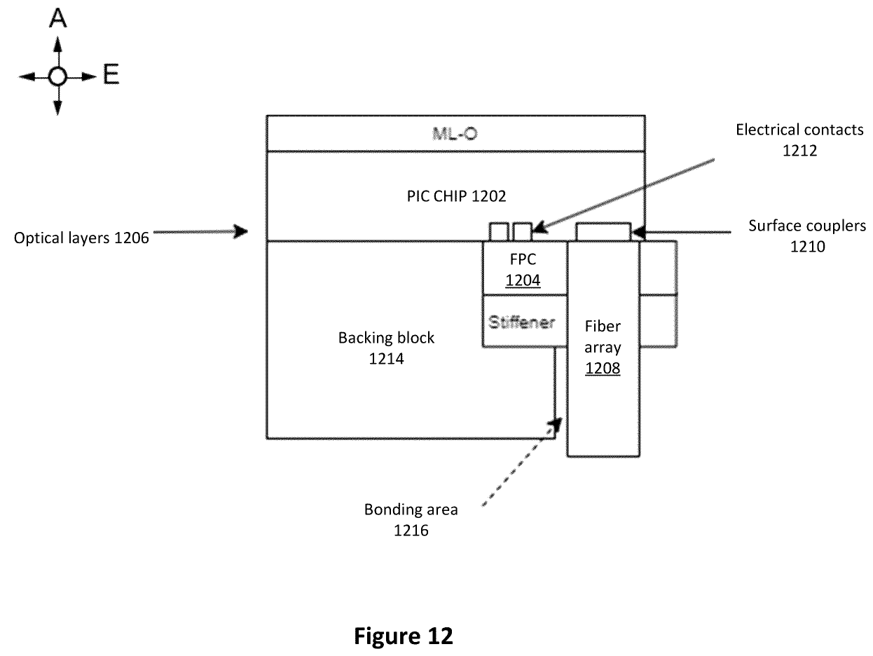

FIG. 2B is an example optical I/O design based on a straight fiber array. As shown in FIG. 2B, the design is based on a straight fiber array 204 coupled to a surface coupler 208 and therefore can realize right-angle bending. Compared to FIG. 2A, the footprint of the fiber array 204 and the interposer chip 210 overlap, and therefore elevation size l_{b}<2 mm could be realized. It should be noted that the interposer chip 210 allows either surface coupler on the top or surface coupler on the bottom (such as by flipping the interposer chip), which does not use through-chip optical coupling from the fiber array 204. The interposer chip 210 can be optional, but the PIC chips usually have optical structures on the top for better/easier acoustic coupling (some of the alternatives discussed herein (e.g., FIG. 12) provide an example where optical structures are not on top of the PIC). Another noteworthy advantage of this design is that it is compatible with a two-dimensional (2D) array of surface couplers (e.g., FIG. 5), which could be combined with (1) multi-core fiber (arrays) or (2) 2D arrays of single-core fibers. In some embodiments, the fiber arrays are a mix of single-mode fiber (“SMF”) and polarization-maintaining fiber (“PMF”).

FIG. 2C is an example optical I/O design based on a vertical interposer chip with a straight fiber array. As shown in FIG. 2C, a vertical interposer chip 210 is coupled to a straight fiber array 204 while also coupled with the edge couplers 206 on the PIC chips via a surface coupler 208. In FIG. 2C, the surface coupler 208 is assumed to be realized with structures in the same optical layers (e.g., silicon, silicon nitride etc.) of waveguide and edge couplers. The right-angle bending of the optical path could also be realized by a 45-degree mirror structure in the vertical interposer chip. The elevational size of the optical I/O is the most compact in this case, e.g., l_{c}<1 mm is possible. However, it should be noted that conventional surface couplers, such as grating couplers (GCs), usually operate with a less-than-right-angle radiation. The most typical angle is 10 degrees from vertical radiation (80-degree bending in this condition). In addition, the radiation pattern of a GC is much larger than that of a waveguide mode. Therefore, specific design of the surface coupler which can enable near-90-degree radiation and compact mode size of the radiation is required for the compact footprint and a high coupling efficiency.

FIG. 2D is an example optical I/O design based on a vertical interposer chip with a mirror structure. As shown in FIG. 2D, the design is based on a vertical interposer chip with a 45-degree mirror structure with a mirror coating 202 that implements a right-angle (e.g., approximately 90 degree) bending. A spot-size converter (optional, not shown) on the vertical interposer chip 210 can be designed close to the mirror structure optimized for the coupling between on-chip optical modes and the waveguide mode of the vertical interposer chip 210. The elevation size of the optical I/O is similar to FIG. 2C, which is determined by the thickness of the vertical interposer chip. In this case, l_{d}<1 mm is possible.

FIG. 2E is an example optical I/O design based on a mirror coating without an interposer chip. As shown in FIG. 2E, the design is the same design as in FIG. 2(a) without the interposer chip 210. In this design, the elevation size of the optical I/O is determined by the thickness of the fiber array 204 of the top part. It should be noted that the drawing is not to scale, and the array definition structure (e.g., V-groove) could be on either side of the PIC module 220. In this way, lₑ could also be very small, for example, lₑ<1 mm is possible.

FIG. 2F is an example optical I/O design based on multiple interposer chips mating with a fiber array. As shown in FIG. 2F, the design varies from the designs in FIG. 2A, but replaces the 45-degree polished fiber array with a fiber array mating with a vertical interposer chip with a 45-degree mirror structure (similar as in FIG. 2D). The elevation size of the optical I/O is determined by the horizontal interposer chip's length and the vertical interposer chip's thickness. l_{f} is similar to lₐ. As shown in FIG. 2F, the design is based on a vertical interposer chip and a horizontal interposer chip. It should be noted that the interposer-PIC chip coupling could benefit from focusing structures on the surface couplers to match the mode mismatch between the radiation mode and the mode in the PIC chip.

FIG. 3 shows another view of FIGS. 2A and 2E with additional details, according to one embodiment. The mode field 302 in FIG. 3 is the core of the fiber array 304. The fiber array 304 includes cladding 314. In the design shown in FIG. 2A, a 45-degree mirror coating 306 is introduced to connect the axial optical path in the fiber array 304 and elevational optical path on interposer/PIC chips on the substrate 312. However, as shown in FIGS. 2A and 2E, without further revision of the fibers, the free-space transmission range, or a coupling gap (d_{gap}), between the axial fiber mode (e.g., fiber array 304) and the elevational waveguide mode (e.g., of the interpose board) is limited above the fiber radius, which is typically 62.5 μm (40 μm, and 25 μm is also available). The spot size converter 310 in the interposer board, on the other hand, is designed to match the mode size difference between the waveguide mode (usually ˜1 μm and the fiber mode (e.g., mode diameter ˜10 μm). An efficient coupling uses a minimal coupling gap (1-5 μm), since a free-space transmission can introduce significant mode diverging that may prohibit the effective mode matching. Therefore, compared to straight fiber array units (FAUs) attached to the interposer chip, the large d_{gap} of design as shown in FIG. 2A could lead to a large insertion loss. It should be noted that once packaged, the gap between the fiber array 304 and the interposer/PIC can be filled with adhesive with a refractive index similar to glass, making the curved boundary of the fiber array nearly invisible to free-space mode after reflection. Thus, little or no focusing effect can be introduced due to the boundary.

A variety of platforms for optical I/O designs may be utilized in various examples. Unique features and limitations in the optical I/O designs, for example as shown in FIGS. 2A-2F, can determine the performance of the optical I/O designs with different platforms. For example, an FAU platform may be utilized. Both one-dimensional (1D) fiber array and 2D fiber array (e.g., muti-core fibers) are available. Fiber diameters (e.g., pitch of 1D fiber array) of 125 (˜127) μm, 80 (˜82) μm and 50 (˜52) μm are available. The mirror coating shown in FIG. 2A is achievable (e.g., using a v-groove and lid structure). Polishing to reduce the cladding between the core and the interposer/PIC is also available, but it can be hard to realize a d_gap<10 μm because (1) of limited polishing accuracy; and/or (2) sufficient cladding thickness to maintain low loss.

Another embodiment utilizes a PIC platform, including but not limited to silicon, silicon nitride integrated photonics platform, which is fabricated with CMOS compatible processes. Compared to the sensor chip, the interposer chip contains simple optical routing with couplers and spot-size converting designs to connect optical modes in different optical waveguides (e.g., fibers to on-chip waveguides). Suspended structures and multi-layer structures could provide flexibility in designing and optimizing couplers.

Yet another embodiment utilizes a glass-based platform (e.g., a planar lightwave circuit). Planar lightwave circuits (PLC) are based on glass materials and can realize a thin and compact footprint thanks to microfabrication processes. While the diversity of optical components available in PLC platforms is not as large as that in PIC platforms, optical routing, spot-size converters, densely distributed couplers, wavelength-division multiplexing (WDM) components, etc. are available. In addition, compared to PIC platforms (based on single crystal substrates and muti-layer CMOS process), PLC platforms have lower cost, shorter lead-time, and more machining flexibility, making it a suitable platform to replace FAUs and interposer chips (PIC platform).

A variety of solutions may be utilized to minimize the coupling gap. For example, and as mentioned above, one way to minimize the coupling gap in FIG. 2A is to polish the fiber cladding down. Such a process likely realizes a coupling gap as small as 10-20 μm because (1) of polishing accuracy and (2) too-thin cladding thickness may lead to significant loss in fibers.

In another embodiment, the root cause of the large coupling gap in design (a) is thick (e.g., tens of micron) cladding in optical fibers. To minimize the gap, the fiber-based structure could be replaced by one or more planar interposers (PIC or PLCs), which could also be polished with an angle and a mirror coating deposited on the facet. This solution could be treated as a variation of the design in FIG. 2C, where the on-chip surface coupler is replaced by an angled mirror plus an in-plane coupler.

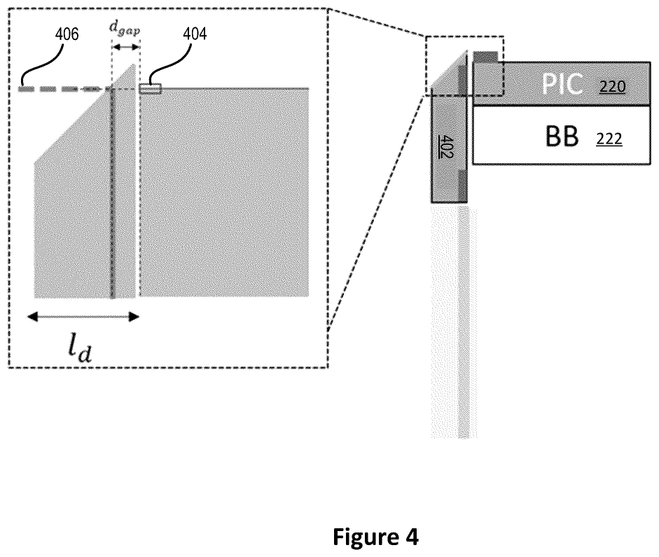

FIG. 4 shows another view of FIG. 2D, illustrating an example interposer with a 45-degree mirror structure, according to one embodiment. It should be noted that horizontal interposer could be added between the vertical interposer 402 and PIC module, as shown in FIG. 2F. The horizontal interposer can include a spot-size converter 404. In FIG. 4, a horizontal dashed line 406 represents a mirror image of waveguides in the horizontal interposer. As shown in FIG. 4, the vertical interposer 402 could be mechanically polished to 45 degrees, and then coated with a mirror. The vertical interposer 402 could be realized in both CMOS-compatible PIC platforms and PLC platforms. A Glass PLC platform is used as an example due to the advantage in cost, lead time, and because arbitrary polishing of glass substrate is widely available. In other examples, a PIC platform can be used, and silicon or silicon nitride can be polished to 45 degrees. During the mirror coating deposition, the transparent facet could be protected by (1) a directional deposition process, (2) a removable protection layer (e.g., a photoresist layer) against the omnidirectional deposition, or (3) allowing for exposure to the omnidirectional deposition but followed by a subsequent polishing. The thickness of the glass lid, which defines the gap size, d_{gap}, is selected to (1) apply minimal impact on the coupling efficiency and (2) facilitate the yield of the mirror coating in front of the spot-size converter 404 (the mirror coating in the gap area serves as a buffer zone for protection layer or post-polishing).

Embodiments of the probe have high laser power design considerations, and thus it may be useful to increase the coupling efficiency. For a high-efficiency, approaching unity, a focusing coupler can be used, since most of the couplers on the chip form a diverging radiation wavefront and unity coupling is the time-reversal of the radiation, i.e., a focusing wavefront. Thus, optical fixtures with right-angle bending and a focusing wavefront based on micro-machining and micro-fabrication rather than nanofabrication of PIC may be utilized. It should be noted the following designs could be realized in fiber FAU platforms, PLC platforms, or PIC platforms.

FIG. 5 is another view of FIG. 2B with a 2D surface coupler array and multi-core fibers in a fiber array, according to one embodiment. As shown in FIG. 5, there are 7 surface couplers 502 forming a 2D surface coupler array. The 2D surface coupler array is coupled with the interposer chip 504 and the fiber array. The fiber array in FIG. 5 includes one or more multi-core fibers 506.

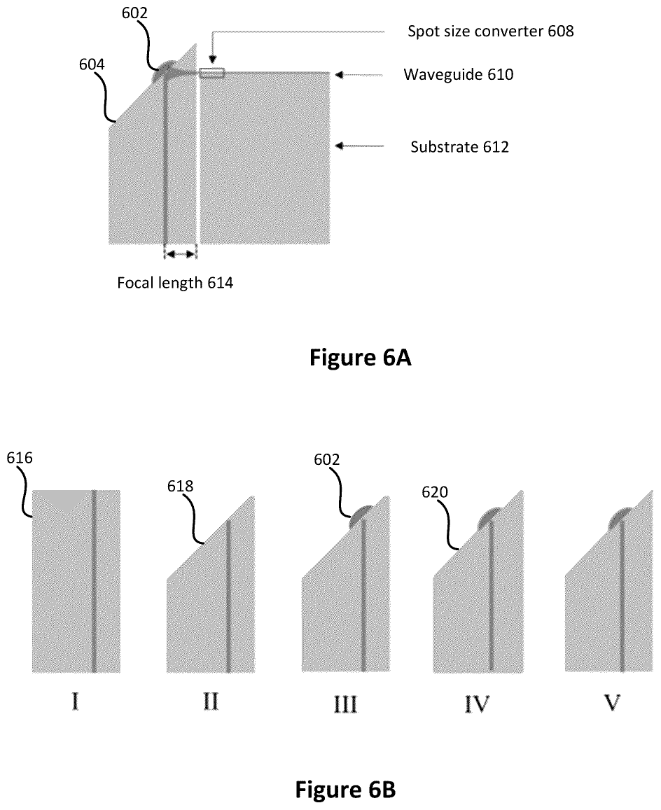

FIG. 6A is an illustration of an embodiment of a reflective focusing right-angle optical fixture, according to one embodiment. FIG. 6B is an example fabrication process of the reflective focusing right-angle optical fixture in FIG. 6A, according to one embodiment. A convex micro-structure 602 could be fabricated on the angled-surface 604 and form a focusing mirror after mirror coating, as shown in FIG. 6A. The spot size converter 608, the waveguide 610, and the substrate can be similar to those in FIG. 3. The fabrication process, as shown in FIG. 6B, starts from an optical waveguide array 616 as shown in step (I), e.g. FAU, interposers. A 45-degree facet 618 is formed by angled polishing as shown in step (II). Then, a convex micro-structure 602 is aligned to the 45-degree facet 618, as shown in step (III), for example by two-photon 3D printing, nanoimprinting, polymer reflow, etc. The whole fixture undergoes mirror layer 620 (e.g., gold layer, dielectric multi-layers . . . ) deposition as shown in step (IV), followed by optical polishing of transparent facets (optional if the mirror coating is directional), as shown in step (V). The focal length of the micro-focusing mirror is realized by controlling the lid thickness. Apart from the focusing enabled by the reflective convex structure, this design has minimal interface or bonding lines between the reflective facet and the on-chip spot size converter, which may be beneficial to minimal interface reflection. However, the shape of the convex needs to be carefully designed and controlled during fabrication, since the curved reflective facet bends and focuses the optical field with a one-time reflection.

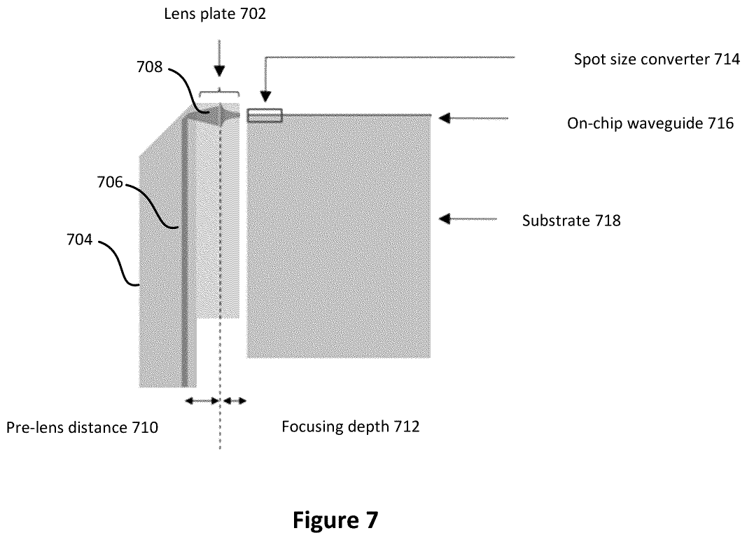

FIG. 7 is an illustration of a refractive focusing right-angle optical fixture with a lens plate, according to one embodiment. While refractive lens structures paired with FAUs are known, existing designs do not provide a flat bonding surface, limiting their application to in-line test rather than optical packaging. The lens is made of a high refractive index material and focuses light into a low refractive index. In FIG. 7, the packaging between off-chip waveguides (e.g., FAUs) and on-chip waveguides uses a flat surface for bonding. The lens may be before or after the 90-degree bend.

The design of a refractive-focusing right-angle optical fixture lens plate 702 located after the 90-degree bend is shown in FIG. 7. The left half of the fixture is the fiber array structure 704 containing waveguide 706 described above, for example as shown in FIG. 4 and FIG. 6A. The right half, or the lens plate 702, could be fabricated separately. The lens 708 is positioned with a pre-lens distance 710 and focusing depth 712 to guide the light into spot size converter 714 and the on-chip waveguides 716 located on substrate 718. It should be noted that an extra low-index coating might be employed to revise a microlens array plate to meet design specifications. As compared to reflective focusing, the fabrication yield of refractive focusing can be higher. In addition, since the lens is aligned to a diverged beam, the alignment tolerance is also larger than that of reflective focusing.

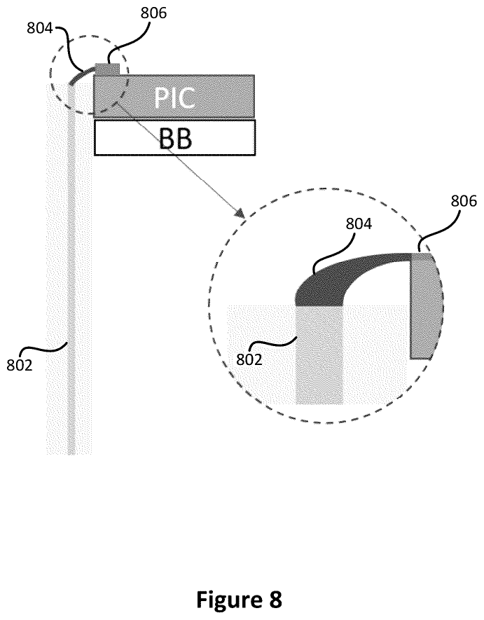

FIG. 8 is an illustration of a design based on photonics wire-bonding techniques in a probe, according to another embodiment. Polymer waveguide (e.g., photonics wire) that provides mode field matching with both optical fiber 802 and edge coupler 806 on PIC chip may be directly fabricated by nanoscale 3D printing. A glob-top or a protective layer can be added to increase the stability of the optical coupling. In this design, the elevation size of the optical I/O is, in principle, roughly the diameter of the fiber. However, this may be affected by the potential limitation from the minimum bending radius of the photonic wire-bonding 804, which could be minimized by the design of waveguide and the materials' index contrast.

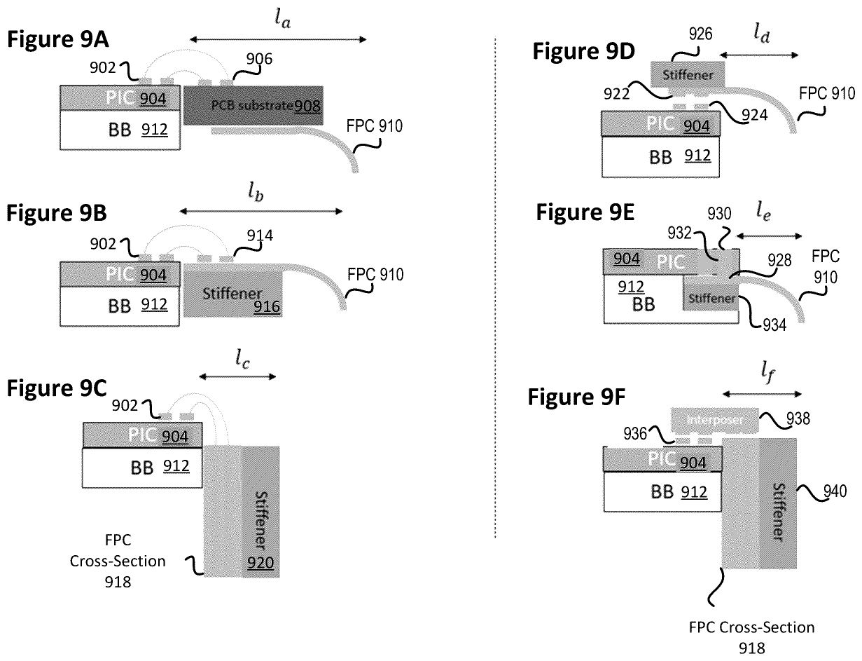

FIGS. 9A-9F show examples of compact electrical I/O designs and packaging for example probes. Design considerations for the electrical I/Os can be similar to that of optical I/Os. However, the electrical contacts can be surface pads on the PIC chips. Thus, the electrical contacts could interleave with each other or form a 2D array, which could reduce the effective pitch of electrical I/O ports. The electrical connections between optical subarray and the flexible printed circuit can be realized with an interposer chip or substrate. Various designs of the electrical I/O are summarized in FIGS. 9A-9F.

FIG. 9A is an example electrical I/O design including 1D pad arrays for wire-bonding a PIC chip and an FPC. As shown in FIG. 9A, the electrical contacts 902 on the PIC chip 904 (supported by the backing block 912) are wire-bonded to the pads 906 on a PCB substrate 908 (e.g., FR-4 printed circuit board (PCB), Aluminum nitride (AlN) substrate, etc.), which connects to the soldering pads on the back side of the PCB substrate through one or more vias (not shown). The FPC 910 is bonded to the backside allowing (1) a larger soldering area, (2) bending within the PCB substrate 908, or (3) a larger pitch of soldering pads because the FPC 910 could overlap with optical I/O in the E-L plane (shown in FIG. 1C). It should be noted that the elevation size lₐ of this design is determined by the footprint of the vias on the PCB substrate 908.

FIG. 9B is another example electrical I/O design including 1D pad arrays for wire-bonding the PIC chip and the FPC. As shown in FIG. 8B, the electrical contacts 902 on the PIC chip 904 are wire-bonded to the bonding pads 914 on FPC 910 that is supported by a stiffener 916 (or the thermomechanical substrate itself). Compared to FIG. 9A, this design uses a high-resolution FPC to match the electrical I/O pitch on the PIC chip 904 and could realize a smaller elevation size l_{b} without the use of vias.

FIG. 9C is yet another example electrical I/O design including 1D pad arrays for wire-bonding the PIC chip and the FPC. As shown in FIG. 9C, the electrical contacts 902 on the PIC chip 904 are wire-bonded to the cross-section 918 of a thick FPC that is bonded to the side of the backing block 912. An ultra-compact elevation footprint with l_{c}<1 mm could be realized. It should be noted that the stiffener 920 could be optional or temporary. The stiffener 920 serves as a hard support for the alignment to the electrical contacts 902 on the PIC chip 904. It can be removed after the electrical link is protected (e.g., with glob top), in some embodiments.

Instead of the wire-bonding process, the examples shown in FIGS. 9D-9F utilize a flip chip bonding process and thus support a 2D pad array. For simplicity, only two rows of pads are shown in the figures.

FIG. 9D is an example electrical I/O design including 2D pad arrays for bonding the PIC chip and the FPC via flip chip bonding. As shown in FIG. 9D, the bonding pads 922 on the FPC 910 are flip chip bonded to the pads 924 on the PIC chip 904, via e.g., soldering bumps or anisotropic conductive paste (ACP), etc. The stiffener 926 could be optional or temporary. After bonding, the stiffener 926 can be removed. If left permanent, the stiffener 926 could serve as a (1) hard protector to provide great robustness to the bonding, (2) electrical isolator, or (3) a flat base structure for further vertical integration.

FIG. 9E is another example electrical I/O design including 2D pad arrays for bonding the PIC chip and the FPC via flip chip bonding. As shown in the example of FIG. 9E, the electrical contacts 928 on the PIC chip 904 are instead on the backside and are linked to the metal traces 930 near the topside by through-silicon vias (TSVs) 932. During assembly, the PIC chip 904 is first bonded to the FPC 910 (via soldering bumps or ACP etc.), and then bonded to the backing block 912 with a minimum bonding line under the sensor region. If needed, the gap between stiffener 934 and the backing block 912 could be filled by underfill material or adhesives.

FIG. 9F is another example electrical I/O design including 2D pad arrays for bonding the PIC chip and the FPC via flip chip bonding. As shown in the example of FIG. 9F, the electrical contacts 936 on the PIC chip 904 and the FPC cross-section 918 are both flip chips bonded to a bridge interposer 938. The stiffener 940 could be optional or temporary.

The elevation size of the three designs in FIGS. 9D-9F is determined by the bending radius and the thickness of FPC, respectively, all of which could be kept under 1 mm. In some examples, the elevation size l for the electrical I/O design above the top surface of PIC chip, for example lₐ, l_{b}, l_{c}, l_{d}, lₑ, and l_{f} in FIGS. 9A-9F can be limited to no more than 0.5 mm for wire-bonding, and no more than 0.2 mm for flip-chip. The elevation size l for optical I/O design, for example lₐ, l_{b}, l_{c}, l_{d}, lₑ, and l_{f} in FIGS. 2A-2F, can be limited to no more than 0.2 mm above top of the PIC chip.

In one embodiment, the probe is manufactured as follows. First, the substrate is attached directly to a flex cable using solder reflow or thermosonic bonding, and underfill can be included as appropriate. Then, the die facet is polished according to whether the die is a right-hand die (“RHD”) or a left-hand die (“LHD”). Dies are then subdiced according to the configuration. Next, one or two dies are mounted to each submount/flex assembly. One or two flake thermistors are also mounted to the respective submount/flex assembly. The dies are then wire-bonded to the AlN submount with glob top. Next, a Peltier cooler is attached to the underside of the AlN submount and the leads are soldered to the AlN submount. Finally, the fiber arrays are attached to the assembly, utilizing the appropriate fixturing.

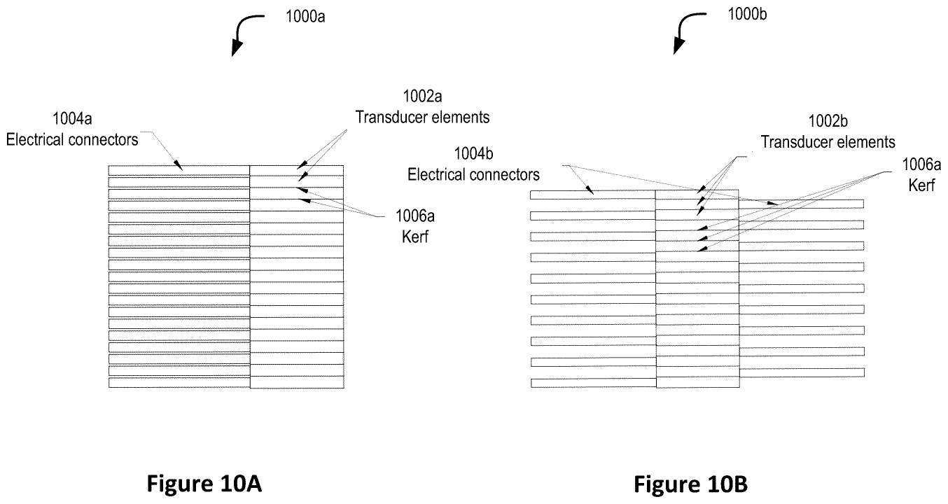

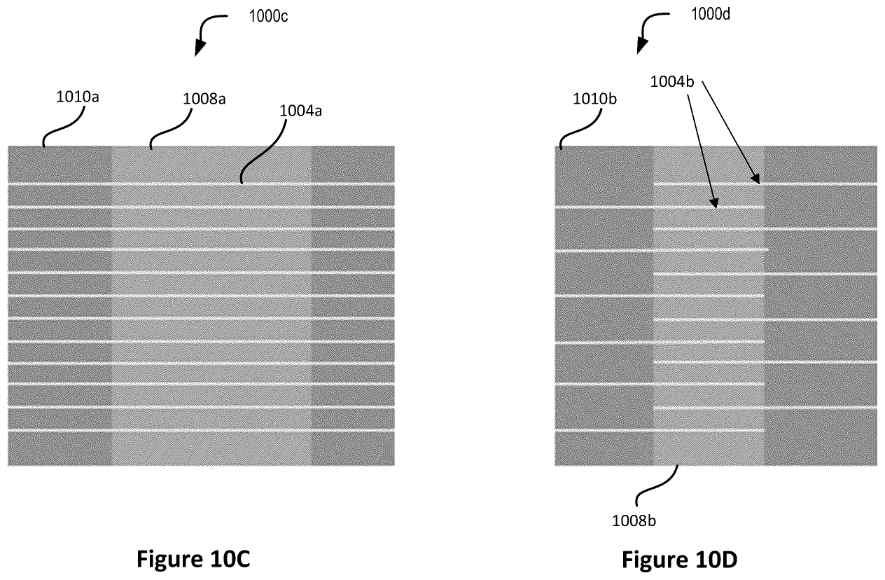

FIG. 10A is an illustration of the design of an AEG module 1000a, according to one embodiment. FIG. 10B is an illustration of a conventional design of an AEG module 1000b, according to one embodiment. In FIG. 10A, the AEG module 1000a is a PZT subarray with transducer elements 1002a, electrical connectors (e.g., electrical wires) 1004a, and kerfs 1006a between the transducer elements 1002a. In FIG. 10b, the AEG module 1000b is a PZT subarray with transducer elements 1002b, electrical connectors (e.g., electrical wires) 1004b, and kerfs 1006b between the transducer elements 1002b. Due to the significant structural changes compared to conventional ultrasound imaging probes and the preference for minimizing the spacing between two subarrays in this novel probe, the design considerations and fabrication process of the PZT subarray have significant differences.

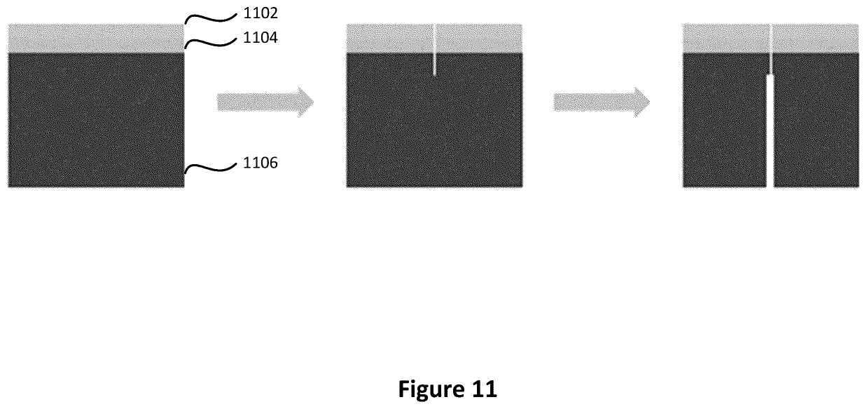

To minimize the gap between the PIC and AEG subarrays, a sharp and clear edge is desired. In conventional ultrasound array fabrication, however, the acoustic stack and circuitry would extend evenly along the elevation direction as shown in FIG. 10B. In the illustrated array design, in order to achieve the AEG array with the circuitry only extending along one side as shown in FIG. 10A the transducer array size was doubled in the elevation direction and a novel process, called “double dicing,” as shown in FIG. 11, can be employed.

FIG. 10C is an illustration of the fabrication of an AEG module 1000c, according to one embodiment. FIG. 10D is an illustration of a conventional fabrication of an AEG module 1000d, according to one embodiment. As shown in FIG. 10C, the transducer array region 1008a in the circuit substrate 1010a maybe doubled and the electrical connectors 1004a cross the whole circuit board, compared to the conventional design in FIG. 10D where electrical connectors 104b alternate from which side they extend to/from transducer array region 1008b. The electrical connectors 104a crossing the whole circuit substrate 1010a can allow the elements to be accessed from both ends of the circuit. The differences in size and connection method are the preparation for the “double dicing” process to achieve clean and sharp edge on the subarray. Thus, prior to dicing, the piezo material is positioned between a backing block and one or more matching layer.

FIG. 11 illustrates a double dicing process, according to one embodiment. The cross-section of the array, for example the AEG modules 1000a shown in FIG. 10a, is shown during the “double dicing” process in FIG. 11. To achieve the edge design considerations, dicing saw with high precision and accuracy is implemented to dice and separate the acoustic stack (matching layers 1102 and piezo material 1104), with the resulting saw kerf slightly extending into the backing block 1106. While the dicing saw could leave an ultra-fine cut surface, nevertheless the clearance of the blade limits the depth of the kerf (e.g., kerf 1006a). The backing block 1106 is an important component in the ultrasound array and its thickness can usually be several millimeters, which prevents the whole array from being separated into two halves by a single cut. Thus, a computer numerical control (CNC) machine can be implemented in this process to cut into the backing block from the bottom. These two cuts can be aligned with fixtures.

Once the “double dicing” process is finished, two symmetric ultrasound arrays of array 1000a shown in FIG. 10A can be acquired. The special circuit design allows access to both arrays with the same pin map and the “double dicing” ensures that both arrays have the clean and sharp edge, which is achieved by dicing saw.

FIGS. 12-14 are illustrations of various example configurations of an I/O packaging design, including a thermomechanical substrate and enclosure for a probe in some embodiments. FIG. 12 illustrates an alternative design of the PIC module package, according to one embodiment. In FIG. 12, the PIC chip 1202 is flip chip bonded to an FPC 1204 via electrical contacts 1212. In FIG. 12, the optical layers 1206 (e.g., optical couplers, waveguides, sensors . . . ) can be considered to be close to the top layers of the PIC chip 1202. FIG. 12 also shows an additional design of the optical I/O. A fiber array 1208, which could be 0-10 degrees away from the vertical angle, could be directly attached to the surface couplers 1210 and thus realize the right-angle bending of optical signals. The fiber array 1208 can also bond to the backing block 1214, with a bonding area 1216.

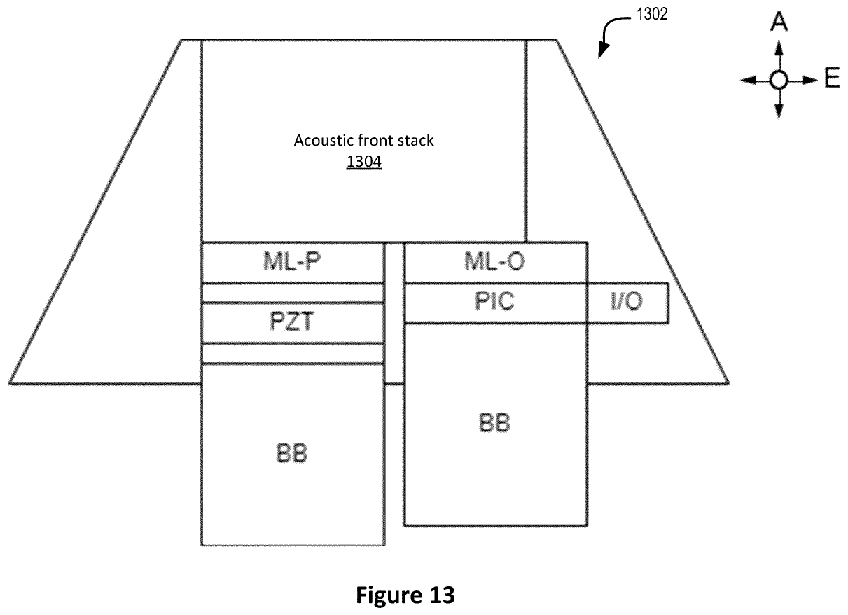

FIG. 13 shows a cone-shaped nosepiece design, according to one embodiment. In some embodiments, the elevation size of the nosepiece is smaller than the designs described above. Thus, a cone-shaped nosepiece 1302 could be introduced as shown in FIG. 13. In FIG. 13, a thick acoustic front stack 1304 lifts the acoustic aperture (not shown) up such that the extra footprint of the I/O structures (both on-chip and off-chip) could be accommodated in the wider base of the nosepiece, while a narrow top interface is realized. The other components shown in FIG. 13 are generally as described in FIG. 1C.

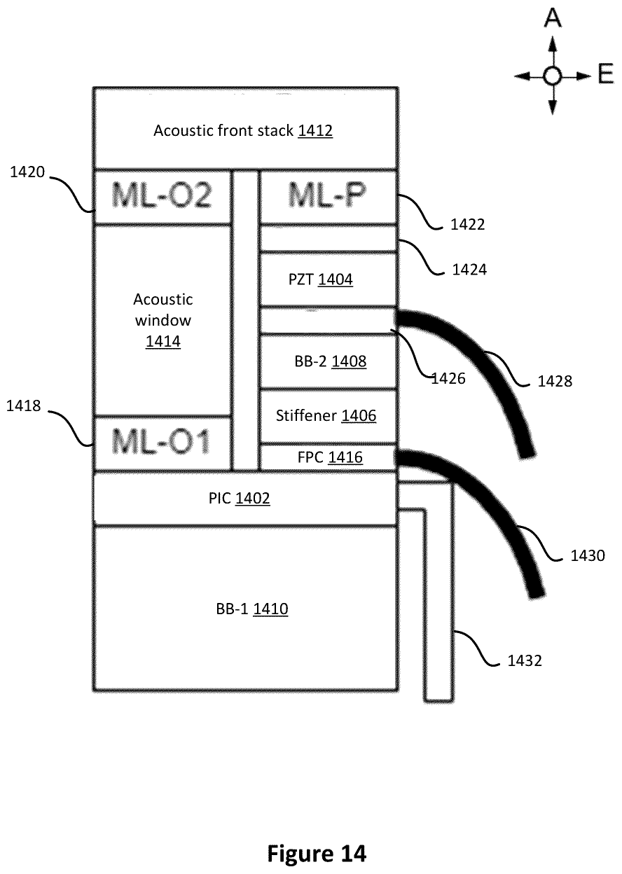

FIG. 14 shows an alternative design of a mixed ultrasound imaging probe, according to one embodiment. In some embodiments where the elevation size of the acoustic aperture (not shown) is so strict that side-by-side PZT and PIC modules are unable to satisfy it, the alternative design shown in FIG. 14 could be introduced. The electrical I/O designs shown in FIGS. 9D and 9E and the alternative design shown in FIG. 9A could provide a flat top surface on PIC module 1402's non-sensing area where the PZT module 1404 could be bonded. The PIC module 1402 is supported by a backing block (e.g., BB-1) 1410. The PIC module 1402 is coupled with optical links 1432 and electrical links 1430. FIG. 14 assumes an electrical I/O design based on the example shown in FIG. 9E. It should be noted that the stiffener 1406 and the BB-2 1408 could be shared by one component, such as made from the same material. For the examples shown in FIGS. 9A and 9E, the stiffener and the FPC are below the PIC. In FIG. 14, the stiffener 1406 and the FPC 1416 are above the PIC module 1402. An acoustic window 1414 can be placed between the acoustic front stack 1412 and the PIC module 1402. There can be a matching layer ML-O2 1418 between the acoustic window 1414 and the PIC module 1402. There can be a matching layer ML-O2 1420 between the acoustic front stack 1412 and the acoustic window 1414. A matching layer ML-P 1422 can be placed between the PZT module 1404 with the acoustic front stack 1412. There can be a conductor layer 1424 between the matching layer ML-P 1422 and the PZT module 1404. The PZT module 1404 can connect to electrical links 1428 via a flex layer 1426.

As with the various other drawings, the size of the elements depicted in FIG. 14 is not representative of the true size of each component in any particular probe. Some embodiments address the offset between the PIC sensor and the PZT ultrasound transducer in the axial direction by implementing a dematching layer into the structure, which may be assembled in between the PZT module 1404 and the flex layer 1426 or between the flex layer 1426 and the BB-2 1408 in FIG. 14. A dematching layer can significantly reduce the thickness of BB-2 1408.

Example probes according to this disclosure can provide one or more advantages. For example, some example probes integrate a PZT ultrasound emitting and receiving system with a PIC ultrasound receiving system while achieving and maintaining the appropriate relative position of the two systems. Such examples further provide mechanical stability for both sensors under normal operating conditions (in conjunction with the housing). Further, such examples efficiently transfer thermal energy away from the PZT and PIC sensor arrays, which can reduce or minimize heat-related degradation in performance (e.g., software “throttling” is not used). Such examples also reduce or minimize the amount of heat transferred to patient and technician.

Moreover, example probes may help to lessen manufacturing costs by, for example, utilizing “design for manufacturing” principles for CNC milling of aluminum components. Further, example probes can be relatively easy to assemble since, for example, fixtures are utilized for proper sensor alignment. Example probes may also help to reduce or minimize the number of parts used to construct the probe. Further, the mixed array subassembly (not including the housing and acoustic lens) can be set securely on a flat surface without additional mechanical support. Such modular designs allow for mechanical assembly of both individual systems to be evaluated individually prior to integration and provides ease of disassembly for servicing and diagnostics.

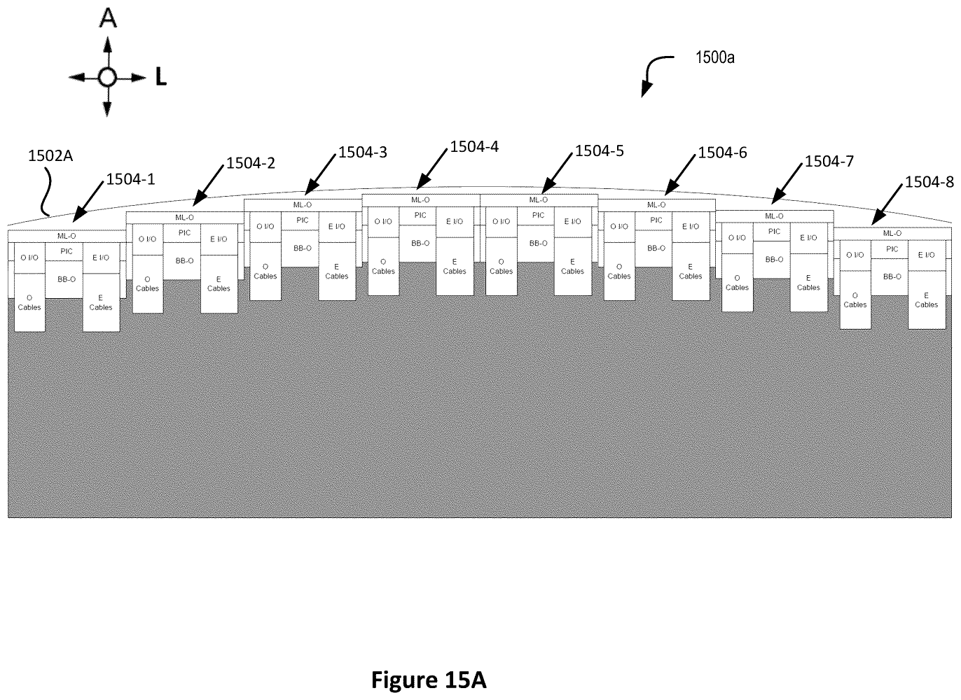

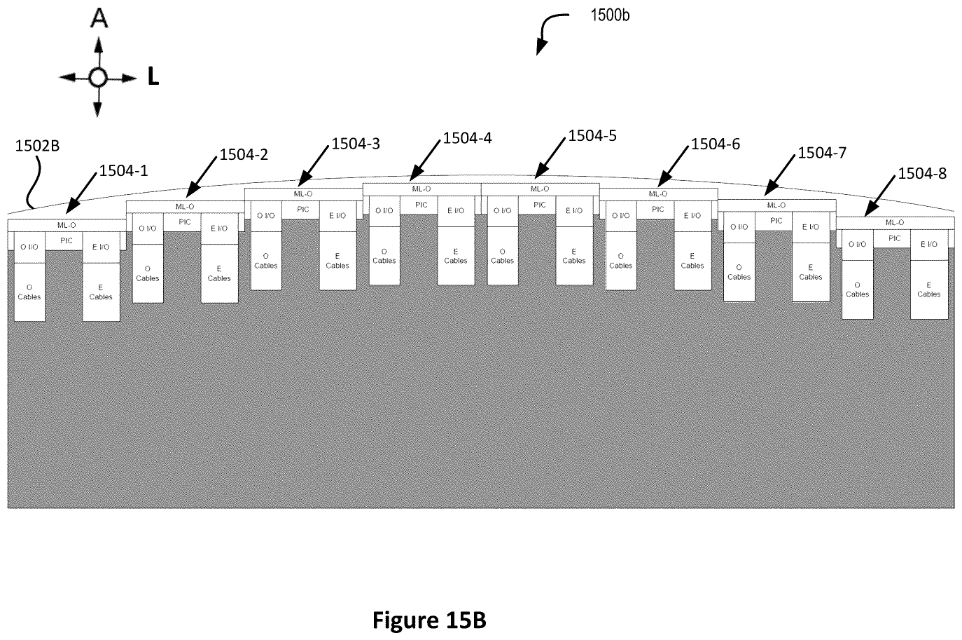

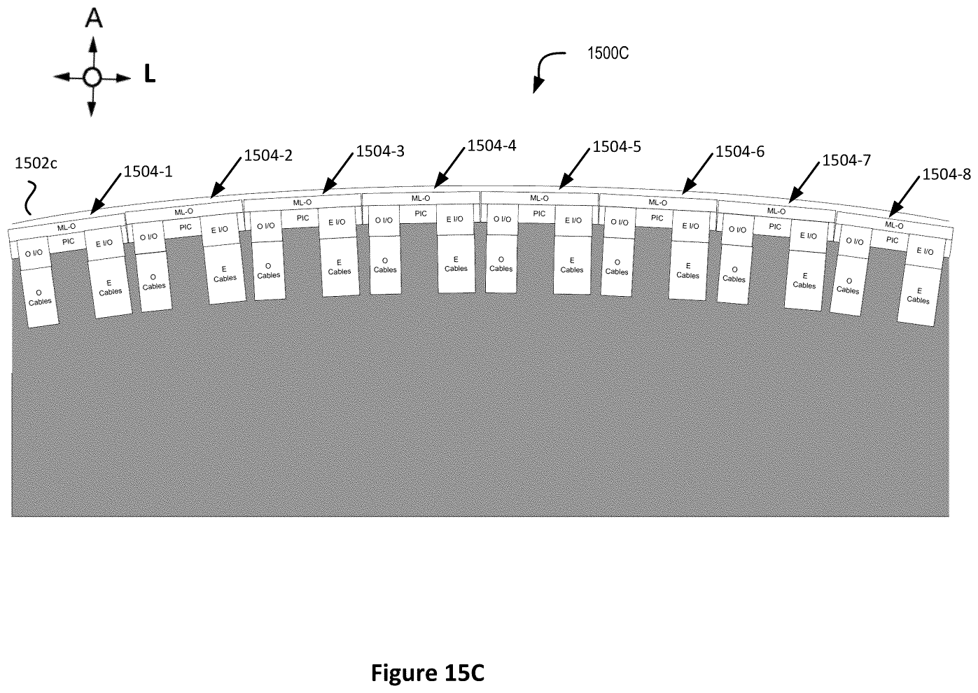

In some embodiments, a curved acoustic front facet is needed, usually known as curvilinear transducers. Given that most PIC platforms have solid substrate, modularized optical subarrays (e.g., modules 154a and 154b) could be introduced to realize curved facets. FIG. 15A is a schematic illustration of modularized optical subarrays arranged in a stair pattern with individual backing blocks, according to one embodiment. FIG. 15B is a schematic illustration of modularized optical subarrays arranged in a stair pattern sharing a unified backing block embedded in a thermomechanical substrate, according to one embodiment. FIG. 15C is a schematic illustration of modularized optical subarrays arranged in a polygonal pattern with individual backing blocks, according to one embodiment.

As shown in FIGS. 15A-15C, 8 optical subarrays (e.g., 1504-1, 1504-2, . . . , 1504-8) are assembled on one thermomechanical substrate, forming a non-flat acoustic receiving window defined by the ML-Os. However, the number of modularized optical subarrays is not limited to 8. The non-flat acoustic receiving window can be a special case of the optical subarray shown in FIG. 1C. The acoustic receiving window 1500A in FIG. 15A and the acoustic receiving window 1500B are both stair-shaped. In contrast, the acoustic receiving window 1500C in FIG. 15C is polygonal.

In FIGS. 15A-15C, a curved acoustic interface (e.g., interface 1502a in FIG. 15A, interface 1502b in FIG. 15B, and interface 1502c in FIG. 2C) covers the acoustic receiving window. The curved acoustic interface could be either shared between the piezo and optical subarrays or independent from that of the piezo subarray.

The acoustic interface could contain multiple layers. For example, interface 1502a in FIG. 15A and interface 1502b in FIG. 15B can contain acoustic windows made of materials with low acoustic attenuation to compensate the interface non-uniform thickness and uniform and thin lens material on the top. In FIG. 15A, each modularized optical subarray contains individual BB-O. In FIG. 15B, each subarray uses PIC as the substrate and shares a unified BB embedded in the thermomechanical substrate.

Similar to FIG. 15B, a unified BB is designed for the polygonal acoustic receiving window shown in FIG. 15C. In general, a cm-level thickness is usually required for BB layer, which can lead to significant gaps between the adjacent acoustic receiving windows.

The foregoing description of example embodiments has been presented only for the purpose of illustration and description and is not intended to be exhaustive or to limit the disclosure to the precise forms disclosed. Numerous modifications and adaptations thereof will be apparent to those skilled in the art without departing from the spirit and scope of the disclosure.

Reference herein to an embodiment, example, or implementation means that a particular feature, structure, operation, or other characteristic described in connection with the example may be included in at least one implementation of the disclosure. The disclosure is not restricted to the particular examples or implementations described as such. The appearance of the phrases “in an embodiment,” “in one example,” “in an example,” “in one implementation,” or “in an implementation,” or variations of the same in various places in the specification does not necessarily refer to the same example or implementation. Any particular feature, structure, operation, or other characteristic described in this specification in relation to one example or implementation may be combined with other features, structures, operations, or other characteristics described in respect of any other example or implementation.

Use herein of the word “or” is intended to cover inclusive and exclusive OR conditions. In other words, A or B or C includes any or all of the following alternative combinations as appropriate for a particular usage: A alone; B alone; C alone; A and B only; A and C only; B and C only; and A and B and C.

1. An apparatus for use in imaging a target, comprising:
A housing; and
a distal portion coupled to the housing, comprising:
an acoustic subarray on a first substrate configured to transmit acoustic signals toward the target;
an optical subarray on a second substrate, configured to detect the acoustic signals from the target; and
an input/output (I/O) region comprising one or more optical I/O channels configured to bend optical signals between the optical subarray and the one or more optical I/O channels, the one or more optical I/O channels comprising an optical fiber array in an axial direction.

2. The apparatus of claim 1, wherein the acoustic subarray comprises acoustic energy generating (AEG) transducers, wherein the AEG transducers comprise one or more of a piezoelectric transducer, a lead zirconate titanate (PZT) transducer, a polymer thick film (PTF) transducer, a polyvinylidene fluoride (PVDF) transducer, a capacitive micromachined ultrasound transducer (CMUT), a piezoelectric micromachined ultrasound transducer (PMUT), a photoacoustic transducer, and a single-crystal transducer.

3. The apparatus of claim 1, wherein the optical subarray comprises one or more photonic integrated circuit (PIC) modules comprising interference-based optical sensors, optical resonators, or interferometers.

4. The apparatus of claim 1, wherein the first substrate comprises aluminum, wherein the acoustic subarray is attached to the first substrate via thermally conductive epoxy.

5. The apparatus of claim 1, wherein the first substrate comprises one or more integrated thermoelectric coolers or one or more thermistors or thermocouples.

6. (canceled)

7. The apparatus of claim 1, wherein the second substrate comprises aluminum or a backing block for the optical subarray.

8. (canceled)

9. The apparatus of claim 1, wherein the housing further comprises a tuning circuit electrically connected to the acoustic subarray, wherein the tuning circuit is configured to condition acoustic signals transmitted from the acoustic subarray.

10. The apparatus of claim 1, wherein the optical fiber array comprises an angled surface with a mirror coating for bending optical signals to and from the optical subarray, wherein the angled surface is about 45 degrees in reference to an elevational-lateral plane of the optical subarray, or the angled surface further comprises a reflective focusing right-angle optical fixture.

11-12. (canceled)

13. The apparatus of claim 1, wherein the one or more optical I/O channels further comprise a refractive focusing right-angle optical fixture with a lens plate, wherein the lens plate is bonded with the optical fiber array and/or an interposer chip via a flat surface or the one or more optical I/O channels further comprises a polymer waveguide bonding the optical fiber array with the optical subarray via one or more edge couplers on the optical subarray.

14. (canceled)

15. The apparatus of claim 1, wherein the optical fiber array is coupled to an interposer chip connecting with the optical subarray in an elevational direction via one or more surface couplers.

16. The apparatus of claim 1, wherein the optical fiber array is mated with an interposer chip in the axial direction comprising a mirror structure with about 45 degrees in reference to an elevational-lateral plane of the optical subarray.

17. The apparatus of claim 1, wherein the I/O region further comprises one or more electrical I/O channels, wherein the one or more electrical I/O channels comprises a flexible printed circuit, wherein the distal portion further comprises an interposer board electrically connected to the optical subarray via the one or more electrical I/O channels.

18. (canceled)

19. The apparatus of claim 17, wherein the flexible printed circuit is connected to the optical subarray via one-dimensional pad array wire-bonding or a two-dimensional pad array flip-chip bonding.

20-23. (canceled)

24. The apparatus of claim 1, further comprising an acoustic front stack with a curved acoustic front facet, wherein the acoustic front stack comprises an interface layer configured to contact a surface of the target for imaging and transmit acoustic signals between the apparatus and the target.

25. (canceled)

26. The apparatus of claim 24, wherein the interface layer comprises a biocompatible material with minimal acoustic impedance, wherein the interface layer comprises at least one of an acoustic matching layer with an acoustic impedance matching with the target for imaging, one or more acoustic lenses configured to focus and steer the acoustic signals to the target, an acoustic window comprising materials with reduced acoustic attenuation and matching acoustic impedance, or an elastomer couplant with low attenuation and impedance.

27-30. (canceled)

31. The apparatus of claim 24, further comprising a first matching layer connecting the interface layer with the acoustic subarray and a second matching layer connecting the interface layer with the optical subarray.

32. The apparatus of claim 1, wherein the housing comprises a type III anodized aluminum housing with a combination of organic contours, dimples, textures, and over-molded silicone regions.

33. The apparatus of claim 1, further comprising a heat sink located in the housing, wherein the heat sink comprises a layer of thermal compound interfacing with the housing.

34. (canceled)

35. The apparatus of claim 1, wherein the one or more optical I/O channels are realized in a fiber array unit (FAU) platform, a PIC platform, or a planar lightwave circuit (PLC) platform.

36-37. (canceled)

38. The apparatus of claim 1, wherein the optical subarray is a flip chip, wherein the optical fiber array is attached to the optical subarray via one or more surface couplers.

39. (canceled)

40. The apparatus of claim 1, wherein the acoustic subarray and the optical subarray are juxtaposed in an elevational direction in the distal portion.

41. The apparatus of claim 1, wherein the acoustic subarray is placed on a flat top surface on a non-sensing area of the optical subarray in the axial direction.

42. The apparatus of claim 1, wherein the optical subarray comprises a plurality of modularized optical subarrays arranged in a stair pattern in an elevational direction or in a polygonal pattern in an elevational direction.

43-46. (canceled)

47. The apparatus of claim 1, wherein a coupling gap between a core of the optical fiber array and a waveguide mode of an interposer in an elevational direction is less than 10 μm.

48. An apparatus for use in imaging a target, comprising:
an acoustic subarray on a first substrate configured to transmit acoustic signals toward the target;
an optical subarray on a second substrate, configured to detect the acoustic signals from the target; and
an input/output (I/O) region comprising one or more optical I/O channels configured to bend optical signals between the optical subarray and the one or more optical I/O channels, the one or more optical I/O channels comprising an optical fiber array in an axial direction.

49-62. (canceled)
